(19) United States
(12) Patent Application Publication
(10) Pub. No.: US 2004/0110204 A1
(43) Pub. Date: Jun. 10, 2004

(54) Compositions, kits,and methods for prognostication, diagnosis, prevention, and treatment of bone-related disorders and other disorders

(75) Inventor: David White, Braintree, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 10/666,807

(22) Filed: Sep. 18, 2003

Related U.S. Application Data
(63) Continuation of application No. 09/628,495, filed on Jul. 28, 2000, now abandoned.
(60) Provisional application No. 60/146,614, filed on Jul. 30, 1999.

(51) Int. Cl. C12Q 1/68

(52) U.S. Cl. 435/6

(57) ABSTRACT

The invention relates to compositions, kits, and methods for predicting, detecting, inhibiting, and alleviating bone-related disorders and other disorders. Examples of bone-related disorders include osteoporosis, Paget's disease, hyperthyroidism, hyperparathyroidism, osteomalacia, chronic renal failure, Cushing's syndrome, osteogenic cancers, and non-osteogenic cancers that have metastasized to bone tissue.

### CROSS-REFERENCE TO RELATED APPLICATIONS

This application is entitled to priority pursuant to 35 U.S.C. §119(e) to U.S. provisional patent application 60/146,614, which was filed on Jul. 30, 1999.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

The invention relates to bone disorders and other disorders, and to compositions, kits, and methods for predicting, detecting, inhibiting, and alleviating these disorders.

G-protein coupled receptors (GPCRs) are a class of proteins responsible for transducing one or more signals from the exterior to the interior of the cell. Examples of extracellular signals include the presence, absence, or concentration of a ligand of a GPCR in the extracellular environment. The intracellular portion of individual GPCRs is capable of interacting with one or more intracellular signaling proteins designated G-proteins.

G-proteins, many of which are trimeric and capable of binding with guanine nucleotides, interact with GPCRs in a specific manner. Upon detection of an extracellular signal (e.g. the presence of a ligand), the GPCR undergoes a change (e.g. a conformational change) which affects its interaction with its corresponding G-protein. Alteration of the GPCR and its corresponding G-protein can have one or more physiological effects within the cell including, for example, alteration of ion channel permeability, alteration of cyclic nucleotide metabolism, modulation of gene transcription, and modulation of enzyme activity. Thus, by means of a GPCR/G-protein pair, a cell is enabled to alter its physiology in response to an extracellular (e.g. environmental or endocrine) stimulus.

G proteins have been extensively described, for example, in Lodish et al. (1995, Molecular Cell Biology, Scientific American Books Inc., New York, N.Y.). GPCRs, G proteins, and G protein-linked effector and second messenger systems have been reviewed in Watson et al. (Eds., 1994, The G-Protein Linked Receptor Fact Book, Academic Press, San Diego, Calif.).

GPCRs and the genes encoding them have been implicated as causative agents of disease (Spiegel et al., 1993, J. Clin. Invest. 92:1119-1125; McKusick et al., 1993, J. Med. Genet. 30:1-26). For example, defects in the rhodopsin gene and in the V2 vasopressin receptor gene have been implicated in retinitis pigmentosum and nephrogenic diabetes insipidus, respectively (Nathans et al., 1992, Annu. Rev. Genet. 26:403-424; Holtzman et al., 1993, Hum. Mol. Genet. 2:1201-1204).

Orthologs of a GPCR of unknown function (designated ‘melatonin-related receptor’ or ‘MRR’) have been identified in humans, rats, and mice (GenBank Accession Nos. U52219, U52218, and AAC21462, respectively; Reppert et al., 1996, FEBS Lett. 386:219-224; Gubitz et al., 1999, Genomics 55:248-251). MRR is encoded by a gene located on the X chromosome in humans and mice (Gubitz et al., 1999, Genomics 55:248-251). A cDNA encoding MRR was originally isolated from a human pituitary cDNA library, and expression of the gene encoding MRR was observed in hypothalamus and pituitary tissues (Reppert et al., 1996, FEBS Lett. 386:219-224). MRR does not bind specifically with melatonin. Until the present invention, MRR was not known to have any physiological function.

Bones form by ossification at membrane surfaces and within cartilage during embryonic development. Bones comprise organic elements, such as fibrous collagen proteins, and mineral elements, such as various mineral salts (primarily calcium and phosphorous salts such as hydroxyapatite). The precise arrangements of proteins and mineral salts within bones varies, depending on the type of bone. Bone cells designated osteoblasts deposit bone tissue, and cells designated osteoclasts resorb bone tissue. In adult animals, bone is continuously deposited and absorbed by the competing actions of osteoblasts and osteoclasts. Uptake of bone material by osteoblasts creates voids within and on the surfaces of bones. Osteoblasts are recruited to these voids and deposit layers of new bone material. In this manner, bone material is continuously regenerated and replenished during life. Osteoblasts which have been largely surrounded by bone material during osteogenesis (i.e., “osteocytes”) remain in communication with one another, and have a role in recruiting osteoclasts to sites of bone stress and injury.

During embryonic development, deposition and resorption of bone tissue are regulated by processes involving genes encoding various proteins (e.g. bone morphogenic proteins, fibroblast growth factor and other growth factors, sonic hedgehog, noggin, Hox, Sox, and the like). Embryonic mesodermal tissue forms buds, which subsequently differentiate to form collagenous tissues containing prechondrocytes and (collagen-producing) chondrocytes. Collagenous bone precursors become vascularized and are invaded by bone-forming osteoblasts and, simultaneously or shortly thereafter, by bone-resorbing osteoclasts, leading to ossification of the precursor and mature bone formation. Each of the phases of bone development, maturation, and homeostasis is regulated by one or more genes, gene products, hormones, and the like, as reviewed in Boyce et al. (1999, Lab. Invest. 79:83-94). Furthermore, the phases of bone development are characterized by the presence and absence of various analytically discernable markers, as reviewed, for example, in Bikle (1997, Am. J. Med. 103:427-436).

Disturbances in normal bone metabolism are associated with a variety of bone-related disorders (i.e. disorders involving aberrant deposition, resorption, or configuration of bone tissue). Examples of bone-related disorders include osteoporosis, Paget's disease, hyperthyroidism, hyperparathyroidism, osteomalacia, chronic renal failure, Cushing's syndrome, and various cancers, including both osteogenic cancers (e.g. osteochondromas and osteogenic sarcomas) and non-osteogenic cancers that have metastasized to bone tissue. These and other bone-related disorders are known in the art, and have been reviewed, for example in Boyce et al. (1999, Lab. Invest. 79:83-94) and Berkow et al. (Eds., 1992, The Merck Manual, Sixth Edition, Merck & Co., Inc., Rahway, N.J.).

Among bone-related disorders, osteoporosis is particularly prevalent, especially among women, including women of advanced age (e.g. post-menopausal women). Osteoporosis is a generalized, progressive diminution of bone density which is generally not accompanied by a significant change in the ratio of mineral elements and organic elements of bone. Osteoporosis is associated with general skeletal weakening, often manifested as a increased incidence of fracture.

Many bone-related disorders are difficult to detect because, among other reasons, easily discernable symptoms are absent until later stages of the disorder. For example, humans afflicted with osteoporosis may be unaware of their condition until one or more bone fractures have occurred. A significant need exists for methods of diagnosing bone-related disorders, and for predicting whether an individual is predisposed to become afflicted with one or more bone-related disorders.

Prior art treatments for bone-related disorders involving diminution of bone mass (e.g. osteoporosis) include administration of anti-resorptive drugs (e.g. estrogen, calcium, vitamin D, calcitonin, and bisphosphonates) and administration of agents that enhance bone formation. Each of these prior art treatments has one or more drawbacks. Estrogen, for example, is associated with enhanced incidence of uterine cancers, breast cancers, or both. Other agents (e.g. calcium and vitamin D) exhibit toxicity at large doses. Other shortcomings of anti-resorptive drugs are that they can inhibit normal mineralization (e.g. as with bisphosphonates) and that they may not enhance bone regeneration in patients having advanced bone diminution. Fluoride (e.g. sodium fluoride) is the only bone formation-enhancing agent that has been widely tested. A significant drawback of fluoride therapy is that bones in patients so treated become unnaturally fragile.

Prior art treatments for bone-related disorders which involve inappropriate localized or general increases in bone mass (e.g. as with certain bone cancers) include treatments generally associated with cancer, such as radiation therapy and chemotherapy. Drawbacks of such therapies are well known and include, for example, insufficient therapeutic specificity (i.e. including both ‘by-stander’ effects and effects attributable to insufficient localization of therapy to diseased tissue).

There is a significant need for compositions and methods for treating bone-related disorders, the compositions and methods preferably not exhibiting the shortcomings of prior art therapies. Also needed are compositions and methods for inhibiting (or, preferably, preventing) bone-related disorders, either prior to the onset of the disorder or early in its progression.

The present invention satisfies the needs identified above.

### SUMMARY OF THE INVENTION

The invention includes several methods of diagnosing a bone-related disorder in a human patient.

A first method of diagnosing a bone-related disorder in a human patient comprises determining the nucleotide sequence of a portion of an mrr gene of the patient and comparing i) the nucleotide sequence of the portion and ii) the corresponding portion of the mrr gene listed in GenBank Accession No. AF003625. A difference between i) the nucleotide sequence of the portion and ii) the corresponding portion of GenBank Accession No. AF003625 is an indication that the patient is afflicted with the bone-related disorder;

A second method of diagnosing a bone-related disorder in a human patient comprises determining the nucleotide sequence of a portion of a transcript polynucleotide of a tissue of the patient, wherein the transcript polynucleotide corresponds to an mrr gene of the patient and comparing i) the nucleotide sequence of the portion and ii) the corresponding portion of SEQ ID NO: 2. A difference between i) the nucleotide sequence of the portion and ii) the corresponding portion of SEQ ID NO: 2 is an indication that the patient is afflicted with the bone-related disorder.

A third method of diagnosing a bone-related disorder in a human patient comprises determining the amino acid sequence of a portion of an MRR protein of a tissue of the patient and comparing i) the amino acid sequence of the portion of the MRR protein of the tissue of the patient and ii) the corresponding portion of SEQ ID NO: 1. A difference between i) the amino acid sequence of the portion of the MRR protein of the tissue of the patient and ii) the corresponding portion of SEQ ID NO: 1 is an indication that the patient is afflicted with the bone-related disorder.

A fourth method of diagnosing a bone-related disorder in a human patient comprises determining the level of expression of an mrr gene in a tissue of the patient and comparing i) the level of expression of the mrr gene in the tissue of the patient and ii) the level of expression of the mrr gene in the same tissue of a human not afflicted with the bone-related disorder. A difference between i) the level of expression of the mrr gene in the tissue of the patient and ii) the level of expression of the mrr gene in the same tissue of the human not afflicted with the bone-related disorder is an indication that the patient is afflicted with the bone-related disorder.

A fifth method of diagnosing a bone-related disorder in a human patient comprises determining the MRR protein content of a tissue of the patient and comparing i) the MRR protein content of the tissue of the patient and ii) the MRR protein content of the same tissue of a human not afflicted with the bone-related disorder. A difference between i) the MRR protein content of the tissue of the patient and ii) the MRR protein content of the same tissue of the human not afflicted with the bone-related disorder is an indication that the patient is afflicted with the bone-related disorder.

A sixth method of diagnosing a bone-related disorder in a human patient comprises administering a detectable MRR-binding agent to the patient, detecting the amount of the MRR-binding agent in the patient, and comparing i) the amount of the MRR-binding agent in the patient and ii) the amount of the MRR-binding agent in a human not afflicted with the bone-related disorder following administration of the MRR-binding agent thereto. A difference between i) the amount of the MRR-binding agent in the patient and ii) the amount of the MRR-binding agent in the human not afflicted with the bone-related disorder following administration of the MRR-binding agent thereto is an indication that the patient is afflicted with the bone-related disorder.

A seventh method of diagnosing a bone-related disorder in a human patient comprises administering a detectable MRR-binding agent to the patient, detecting the amount of the MRR-binding agent associated with a bone tissue in the patient, and comparing i) the amount of the MRR-binding agent associated with a bone tissue in the patient and ii) the amount of the MRR-binding agent associated with a bone tissue in a human not afflicted with the bone-related disorder following administration of the MRR-binding agent thereto. A difference between i) the amount of the MRR-binding agent associated with a bone tissue in the patient and ii) the amount of the MRR-binding agent associated with a bone tissue in the human not afflicted with the bone-related disorder following administration of the MRR-binding agent thereto is an indication that the patient is afflicted with the bone-related disorder.

In these methods of diagnosing a bone-related disorder in a human patient, the bone-related disorder can, for example, be selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue. The tissue of the patient can be a bone tissue or a tissue sample obtained from the patient, such as a bone tissue biopsy, a blood fluid, or a cerebrospinal fluid. Also the human not afflicted with the bone-related disorder can be a consensus profile of humans not afflicted with the bone-related disorder. In these methods, the transcript polynucleotide can, for example, be an mRNA or a cDNA (e.g. a cDNA consisting of the entire open reading frame corresponding to the cDNA).

In these methods of diagnosing a bone-related disorder in a human patient, the MRR-binding agent can be an antibody substance, such as one attached to an imaging agent. The amount of the MRR-binding agent can, for example, be detected using an imaging method in which the imaging agent can be detected. The mrr gene listed in GenBank Accession No. AF003625 preferably includes nucleotide residues 27599 through 32411 of GenBank Accession No. AF003625. The portion of the MRR protein used in these methods can be the entire amino acid sequence of the MRR protein.

In another aspect, the invention also includes several methods of determining whether an individual is predisposed to become afflicted with a bone-related disorder.

A first method of determining whether an individual is predisposed to become afflicted with a bone-related disorder comprises determining the nucleotide sequence of a portion of an mrr gene of the patient and comparing i) the nucleotide sequence of the portion and ii) the corresponding portion of the mrr gene listed in GenBank Accession No. AF003625. A difference between i) the nucleotide sequence of the portion and ii) the corresponding portion of GenBank Accession No. AF003625 is an indication that the patient is predisposed to become afflicted with the bone-related disorder.

A second method of determining whether an individual is predisposed to become afflicted with a bone-related disorder comprises determining the nucleotide sequence of a portion of a transcript polynucleotide of a tissue of the patient, wherein the transcript polynucleotide corresponds to an mrr gene of the patient and comparing i) the nucleotide sequence of the portion and ii) the corresponding portion of SEQ ID NO: 2. A difference between i) the nucleotide sequence of the portion and ii) the corresponding portion of SEQ ID NO: 2 is an indication that the patient is predisposed to become afflicted with the bone-related disorder.

A third method of determining whether an individual is predisposed to become afflicted with a bone-related disorder comprises determining the amino acid sequence of a portion of an MRR protein of a tissue of the patient and comparing i) the amino acid sequence of the portion of the MRR protein of the tissue of the patient and ii) the corresponding portion of SEQ ID NO: 1. A difference between i) the amino acid sequence of the portion of the MRR protein of the tissue of the patient and ii) the corresponding portion of SEQ ID NO: 1 is an indication that the patient is predisposed to become afflicted with the bone-related disorder.

In these methods of determining whether an individual is predisposed to become afflicted with a bone-related disorder, the bone-related disorder can, for example, be selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue.

The invention further relates to several methods of alleviating a bone-related disorder in a human patient, such as a bone-related disorder selected from the group consisting of osteoporosis, Paget's disease, hyperthyroidism, hyperparathyroidism, osteomalacia, chronic renal failure, Cushing's syndrome, an osteogenic cancer, or a non-osteogenic cancer that has metastasized to bone tissue. These methods include:

providing a biologically active portion of an MRR protein (e.g. protein having the sequence SEQ ID NO: 1) to a bone tissue of the patient in an amount sufficient to alleviate the bone-related disorder;

providing a non-functional MRR protein to a bone tissue of the patient in an amount sufficient to alleviate the bone-related disorder;

providing an expression vector comprising a polynucleotide encoding a biologically active portion of an MRR protein (e.g. polynucleotide having the sequence SEQ ID NO: 2) to a bone tissue of the patient in an amount sufficient to alleviate the bone-related disorder;

providing an expression vector comprising a polynucleotide encoding a non-functional MRR protein to a bone tissue of the patient in an amount sufficient to alleviate the bone-related disorder;

providing an agonist of MRR protein activity to a bone tissue of the patient in an amount sufficient to alleviate the bone-related disorder;

providing an antagonist of MRR protein activity (e.g. an antibody substance which binds specifically with MRR protein) to a bone tissue of the patient in an amount sufficient to alleviate the bone-related disorder;

providing an enhancer of mrr expression to a bone tissue of the patient in an amount sufficient to alleviate the bone-related disorder; and

providing an inhibitor of mrr expression (e.g. an anti-sense oligonucleotide or a ribozyme) to a bone tissue of the patient in an amount sufficient to alleviate the bone-related disorder.

In yet another aspect, the invention includes several methods of inhibiting a bone-related disorder in a human patient at risk for developing the bone-related disorder such as osteoporosis, Paget's disease, hyperthyroidism, hyperparathyroidism, osteomalacia, chronic renal failure, Cushing's syndrome, an osteogenic cancer, or a non-osteogenic cancer that has metastasized to bone tissue. These methods include:

providing a biologically active portion of an MRR protein to a bone tissue of the patient in an amount sufficient to inhibit the bone-related disorder;

providing a non-functional MRR protein to a bone tissue of the patient in an amount sufficient to inhibit the bone-related disorder;

providing an expression vector comprising a polynucleotide encoding a biologically active portion of an MRR protein to a bone tissue of the patient in an amount sufficient to inhibit the bone-related disorder;

providing an expression vector comprising a polynucleotide encoding a non-functional MRR protein to a bone tissue of the patient in an amount sufficient to inhibit the bone-related disorder;

providing an agonist of MRR protein activity to a bone tissue of the patient in an amount sufficient to inhibit the bone-related disorder;

providing an antagonist of MRR protein activity to a bone tissue of the patient in an amount sufficient to inhibit the bone-related disorder;

providing an enhancer of mrr expression to a bone tissue of the patient in an amount sufficient to inhibit the bone-related disorder; and

providing an inhibitor of mrr expression to a bone tissue of the patient in an amount sufficient to inhibit the bone-related disorder.

In still another aspect, the invention includes several methods of determining whether a test composition is useful for alleviating a bone-related disorder such as osteoporosis, Paget's disease, hyperthyroidism, hyperparathyroidism, osteomalacia, chronic renal failure, Cushing's syndrome, an osteogenic cancer, or a non-osteogenic cancer that has metastasized to bone tissue.

A first method of determining whether a test composition is useful for alleviating a bone-related disorder comprises maintaining a cell which comprises a biologically active MRR protein (e.g. a protein having the amino acid sequence SEQ ID NO: 1) in the presence of the test composition and comparing i) an activity of the MRR protein of the cell maintained in the presence of the test composition (e.g. an activity selected from the group consisting of a proteolytic activity, a pore-modulating activity, an enzyme-modulating activity, and a gene transcription-modulating activity) and ii) the same activity of the MRR protein of a cell of the same type maintained in the absence of the test composition. A difference between i) an activity of the MRR protein of the cell maintained in the presence of the test composition and ii) the same activity of the MRR protein of the cell of the same type maintained in the absence of the test composition is an indication that the test composition is useful for alleviating a bone-related disorder.

A second method of determining whether a test composition is useful for alleviating a bone-related disorder comprises maintaining a cell which comprises a biologically active MRR protein in the presence of the test composition and comparing i) the level of expression of the mrr gene in the cell maintained in the presence of the test composition and ii) the level of expression of the mrr gene in the MRR protein of a cell of the same type maintained in the absence of the test composition. A difference between i) the level of expression of the mrr gene in the cell maintained in the presence of the test composition and ii) the level of expression of the mrr gene in the MRR protein of a cell of the same type maintained in the absence of the test composition is an indication that the test composition is useful for alleviating a bone-related disorder.

A third method of determining whether a test composition is useful for alleviating a bone-related disorder compresses maintaining a cell which comprises a biologically active MRR protein in the presence of the test composition and comparing i) a bone phenotype of the cell maintained in the presence of the test composition and ii) the same bone phenotype of a cell of the same type maintained in the absence of the test composition. A difference between i) the bone phenotype (e.g. a phenotype selected from the group consisting of a bone deposition phenotype, a bone resorption phenotype, and a bone morphology phenotype) of the cell maintained in the presence of the test composition and ii) the same bone phenotype of the cell maintained in the absence of the test composition is an indication that the test composition is useful for alleviating a bone-related disorder.

A fourth method of determining whether a test composition is useful for alleviating a bone-related disorder compresses administering the test composition to a first animal which naturally harbors an mrr gene (e.g. an mrr gene selected from the group consisting of a human mrr gene, a mouse mrr gene, and a rat mrr gene, or one which encodes a protein having an amino acid sequence selected from the group consisting of SEQ ID NOs: 1 and 3) and comparing i) a bone phenotype of the first transgenic animal and ii) the same bone phenotype of a second animal which naturally harbors an mrr gene and to which the test composition is not administered. A difference between i) the bone phenotype of the first animal and ii) the same bone phenotype of the second animal is an indication that the test composition is useful for alleviating a bone-related disorder.

A fifth method of determining whether a test composition is useful for alleviating a bone-related disorder comprises administering the test composition to a first non-human transgenic animal which harbors an exogenous mrr gene (e.g. one which protein having the amino acid sequence SEQ ID NO: 1) and comparing i) a bone phenotype of the first transgenic animal and ii) the same bone phenotype of a second non-human transgenic animal which harbors an exogenous mrr gene and to which the test composition is not administered. A difference between i) the bone phenotype of the first transgenic animal and ii) the same bone phenotype of the second transgenic animal is an indication that the test composition is useful for alleviating a bone-related disorder.

A sixth method of determining whether a test composition is useful for alleviating a bone-related disorder compresses maintaining an artificial membrane (e.g. a liposome or a re-sealed erythrocyte) which comprises a biologically active MRR protein in the presence of the test composition and comparing i) an activity of the MRR protein of the artificial membrane maintained in the presence of the test composition and ii) the same activity of the MRR protein of an artificial membrane of the same type maintained in the absence of the test composition> A difference between i) the activity of the MRR protein of the artificial membrane maintained in the presence of the test composition and ii) the same activity of the MRR protein of artificial membrane of the same type maintained in the absence of the test composition is an indication that the test composition is useful for alleviating a bone-related disorder.

The cell used in these methods of determining whether a test composition is useful for alleviating a bone-related disorder is preferably an animal cell, including a bone cell. For example, the cell can be selected from the group consisting of a human cell, a mouse cell, and a rat cell.

The invention also includes several methods of determining the propensity of a test compound to induce a bone-related disorder in a human patient (e.g. osteoporosis, Paget's disease, hyperthyroidism, hyperparathyroidism, osteomalacia, chronic renal failure, Cushing's syndrome, an osteogenic cancer, or a non-osteogenic cancer that has metastasized to bone tissue).

A first method of determining the propensity of a test compound to induce a bone-related disorder comprises maintaining a cell which comprises a biologically active MRR protein in the presence of the test composition and comparing i) an activity of the MRR protein of the cell maintained in the presence of the test composition and ii) the same activity of the MRR protein of a cell of the same type maintained in the absence of the test composition. A difference between i) an activity of the MRR protein of the cell maintained in the presence of the test composition and ii) the same activity of the MRR protein of the cell of the same type maintained in the absence of the test composition is an indication that the test composition is likely to induce the bone-related disorder in a human patient.

A second method of determining the propensity of a test compound to induce a bone-related disorder comprises maintaining a cell which comprises a biologically active MRR protein in the presence of the test composition and comparing i) the level of expression of the mrr gene in the cell maintained in the presence of the test composition and ii) the level of expression of the mrr gene in the MRR protein of a cell of the same type maintained in the absence of the test composition. A difference between i) the level of expression of the mrr gene in the cell maintained in the presence of the test composition and ii) the level of expression of the mrr gene in the MRR protein of a cell of the same type maintained in the absence of the test composition is an indication that the test composition is likely to induce the bone-related disorder in a human patient.

A third method of determining the propensity of a test compound to induce a bone-related disorder comprises maintaining a cell which comprises a biologically active MRR protein in the presence of the test composition and comparing i) a bone phenotype of the cell maintained in the presence of the test composition and ii) the same bone phenotype of a cell of the same type maintained in the absence of the test composition. A difference between i) the bone phenotype of the cell maintained in the presence of the test composition and ii) the same bone phenotype of the cell maintained in the absence of the test composition is an indication that the test composition is likely to induce the bone-related disorder in a human patient.

A fourth method of determining the propensity of a test compound to induce a bone-related disorder comprises administering the test composition to a first animal which naturally harbors an mrr gene and comparing i) a bone phenotype of the first transgenic animal and ii) the same bone phenotype of a second animal which naturally harbors an mrr gene and to which the test composition is not administered. A difference between i) the bone phenotype of the first animal and ii) the same bone phenotype of the second animal is an indication that the test composition is likely to induce the bone-related disorder in a human patient.

A fifth method of determining the propensity of a test compound to induce a bone-related disorder comprises administering the test composition to a first non-human transgenic animal which harbors an exogenous mrr gene and comparing i) a bone phenotype of the first transgenic animal and ii) the same bone phenotype of a second non-human transgenic animal which harbors an exogenous mrr gene and to which the test composition is not administered. A difference between i) the bone phenotype of the first transgenic animal and ii) the same bone phenotype of the second transgenic animal is an indication that the test composition is likely to induce the bone-related disorder in a human patient.

A sixth method of determining the propensity of a test compound to induce a bone-related disorder comprises maintaining an artificial membrane which comprises a biologically active MRR protein in the presence of the test composition and comparing i) an activity of the MRR protein of the artificial membrane maintained in the presence of the test composition and ii) the same activity of the MRR protein of an artificial membrane of the same type maintained in the absence of the test composition. A difference between i) an activity of the MRR protein of the artificial membrane maintained in the presence of the test composition and ii) the same activity of the MRR protein of artificial membrane of the same type maintained in the absence of the test composition is an indication that the test composition is likely to induce the bone-related disorder in a human patient.

In addition, the invention includes a method of identifying a polymorphism associated with an mrr gene of a human patient afflicted with a bone-related disorder such as osteoporosis, Paget's disease, hyperthyroidism, hyperparathyroidism, osteomalacia, chronic renal failure, Cushing's syndrome, an osteogenic cancer, or a non-osteogenic cancer that has metastasized to bone tissue. This method comprises determining the nucleotide sequence of a polynucleotide associated with the mrr gene in the patient and comparing i) the nucleotide sequence of a polynucleotide associated with the mrr gene in the patient and ii) the nucleotide sequence of the corresponding portion of an mrr gene of a human not afflicted with the bone-related disorder. A difference between i) the nucleotide sequence of the polynucleotide and ii) the nucleotide sequence of the corresponding portion of an mrr gene of the human not afflicted with the bone-related disorder indicates a polymorphism associated with the mrr gene of the human patient afflicted with the bone-related disorder.

The invention further relates to a method of determining whether a human patient is predisposed to become afflicted with a bone-related disorder. The method comprises determining whether a polymorphism identified by a method described herein occurs in an mrr gene of the human patient. Occurrence of the polymorphism in the mrr gene of the human patient is an indication that the human patient is predisposed to become afflicted with the bone-related disorder.

### BRIEF DESCRIPTION OF THE SEVERAL VIEWS OF THE DRAWINGS

FIG. 1, comprising FIGS. 1A and 1B, is an alignment of the amino acid sequences of human (H; SEQ ID NO: 1), murine (M; SEQ ID NO: 3), and rat (R; SEQ ID NO: 5) MRR proteins. The rat sequence is a partial protein sequence.

FIG. 2, comprising FIGS. 2A, 2B, and 2C, lists the nucleotide sequences corresponding to human (FIG. 2A; SEQ ID NO: 2), murine (FIG. 2B; SEQ ID NO: 4), and rat (FIG. 2C; SEQ ID NO: 6) MRR proteins.

FIG. 3 is an image generated in a in situ hybridization experiment in which mRNA encoding MRR protein was detected in a whole mount of an 18.5-day murine embryo. MRR-encoding mRNA is indicated by dark shading. The presence of MRR-encoding mRNA is evident in, for example, vertebral and limb bone precursors in the embryo.

FIG. 4, comprising FIGS. 4A, 4B, 4C, and 4D, is a quartet of images of 18.5-day murine embryonic limb bone precursor (FIGS. 4A and 4B) and vertebral disk bone precursor (FIGS. 4C and 4D). FIGS. 4A and 4C are images of the microscopic appearance of these tissues, and FIGS. 4B and 4D depict the presence of MRR protein, as assessed by fluorescent in situ hybridization using an antibody which binds specifically with MRR.

FIG. 5, comprising FIGS. 5A through 51, is an alignment of the nucleotide sequence (SEQ ID NO: 2; GenBank Accession No. U52219) of the cDNA encoding human MRR protein and residues 27599-27855 (SEQ ID NO: 7), 30712-32031 (SEQ ID NO: 8), and 32044-32411 (SEQ ID NO: 9) of a cosmid derived from human chromosome X genomic DNA (GenBank Accession No. AF003625). The amino acid sequence (“MRR AA”; SEQ ID NO: 1) is listed beneath the cDNA sequence for reference.

### DETAILED DESCRIPTION OF THE INVENTION

The present invention is based on the discovery that a G-protein coupled receptor designated melatonin-related receptor (MRR), which is expressed in hypothalamus and pituitary tissues in humans and which is also expressed in mouse and rat tissues, is involved in one or more bone-related disorders. As described in the Background section of this application, others have identified nucleic acid sequences which encode MRR in humans and mice, and a partial rat sequence for MRR has also been reported. Available evidence indicates that MRR is broadly conserved among mammals, and likely among all vertebrates.

Previous reports described expression of MRR in hypothalamus and pituitary tissues. However, it was not previously recognized that MRR is expressed in bone tissue, including embryonic bone tissue. Thus, until the present invention, it was not recognized the MRR is involved in one or more bone-related disorders such as osteoporosis, Paget's disease, hyperthyroidism, hyperparathyroidism, osteomalacia, chronic renal failure, Cushing's syndrome, osteogenic cancers, and non-osteogenic cancers that have metastasized to bone tissue.

The present invention relates to methods involving human, murine, rat, and other mammalian and vertebrate MRR proteins, fragments, and variants of these proteins, as described below. These proteins, fragments, and variants are, individually and collectively, referred to herein as “polypeptides of the invention.” Polynucleotides (i.e. including genomic DNA, cDNA, mRNA, and other polynucleotides) which encode one or more polypeptides of the invention are, individually and collectively, referred to herein (interchangeably) as “nucleic acids of the invention,” “nucleic acid molecules of the invention,” and “polynucleotides of the invention.”

Diagnostic Methods for Bone-Related Disorders

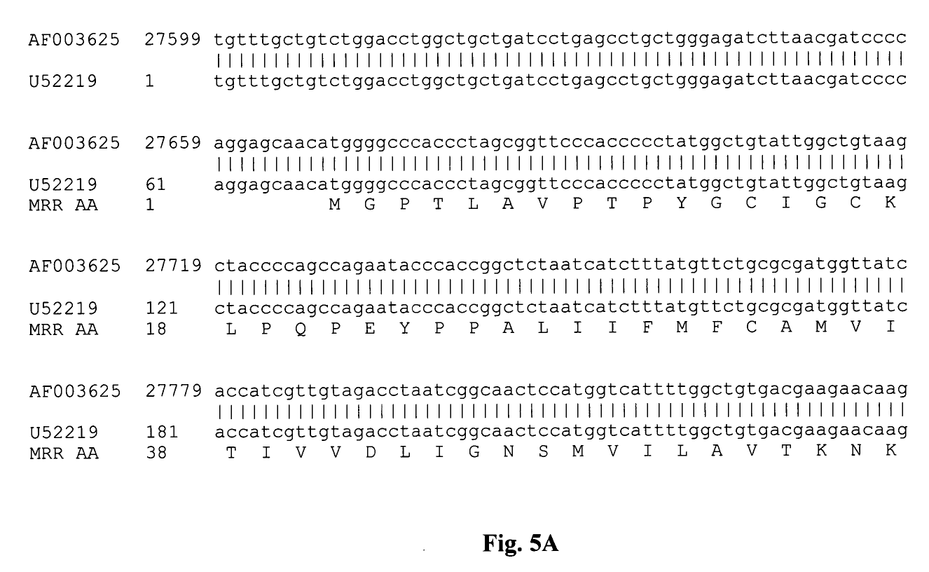

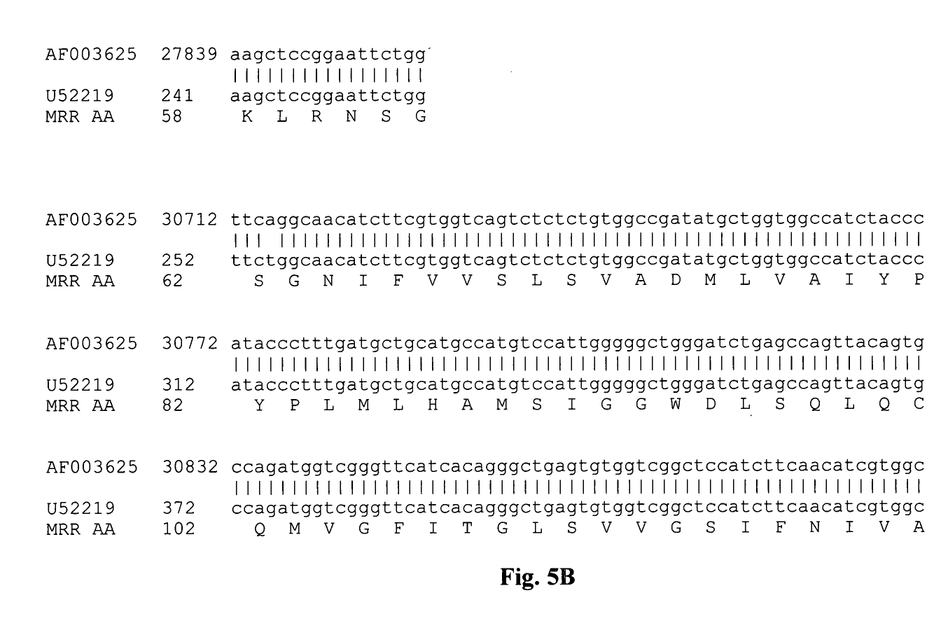

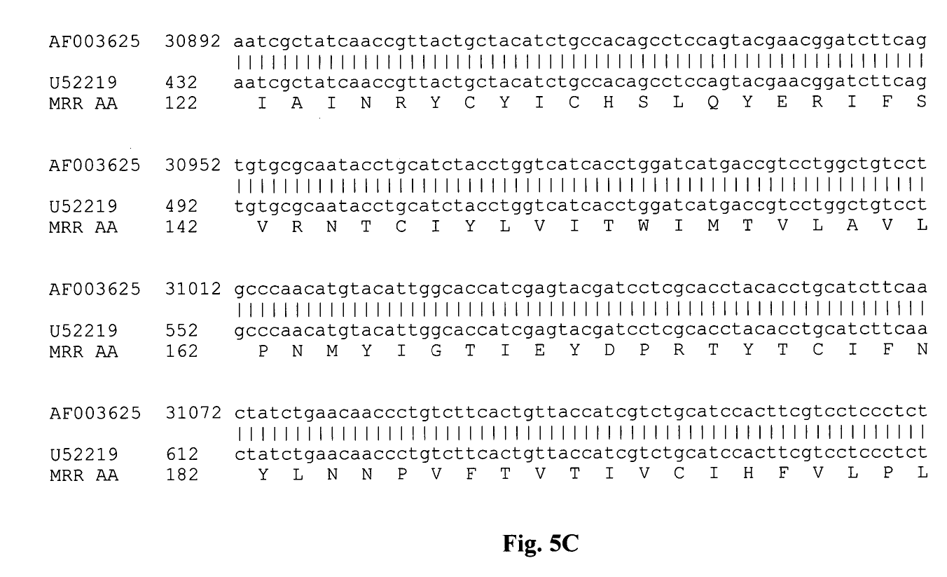

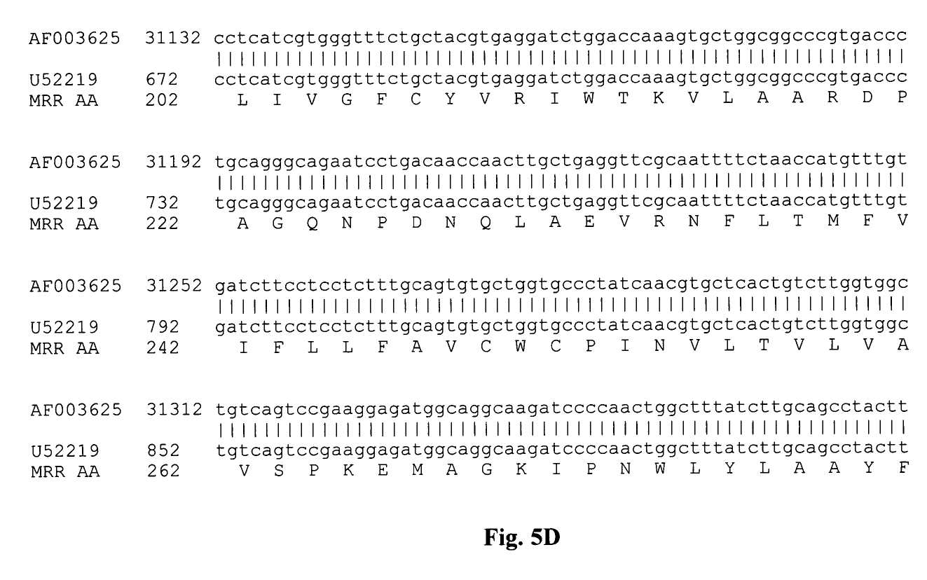

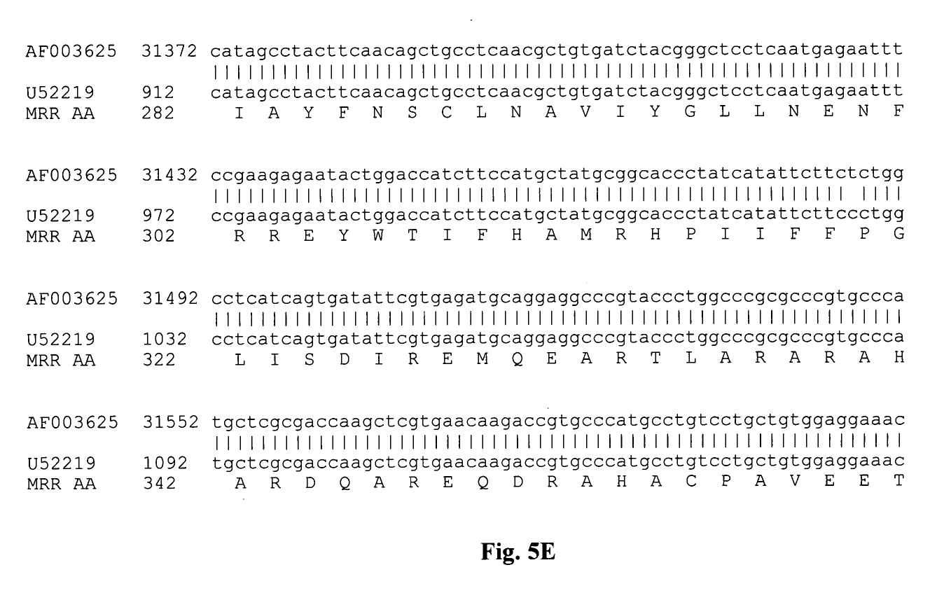

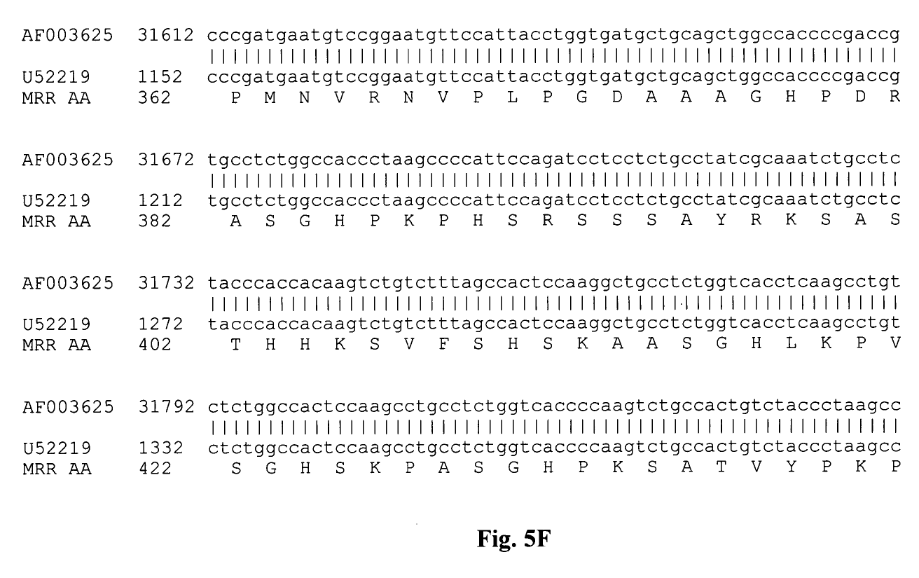

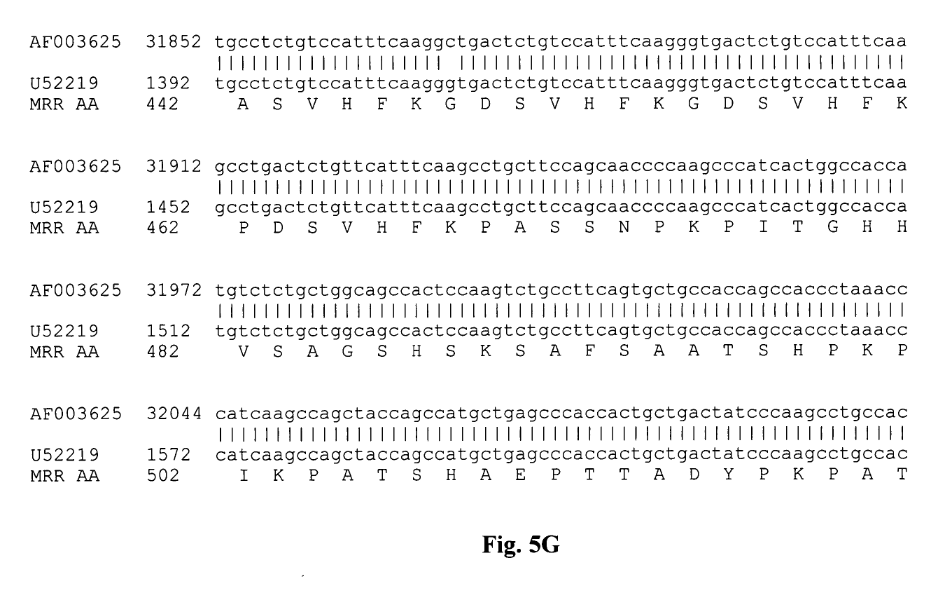

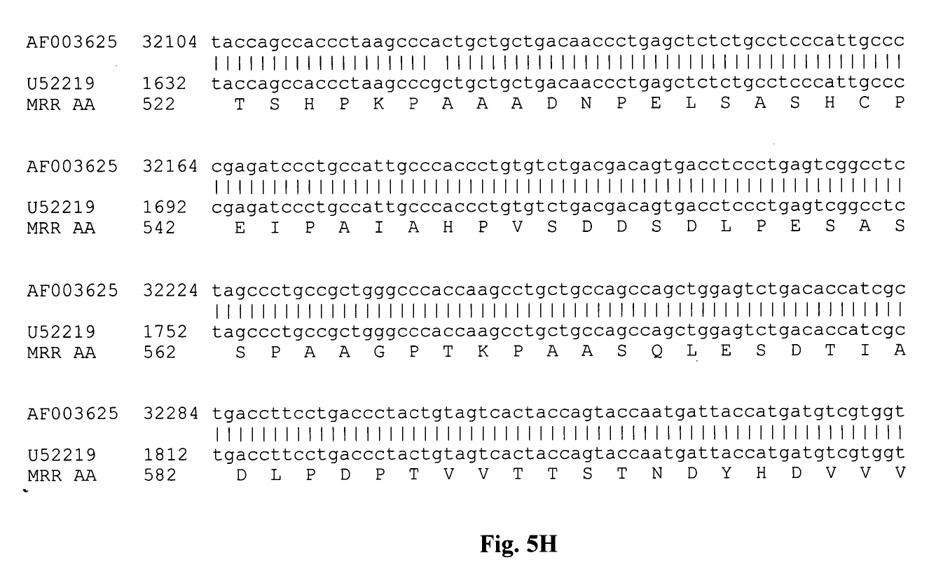

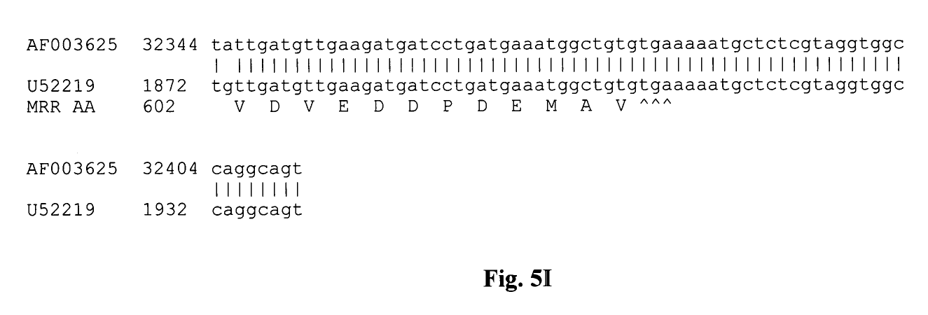

The invention includes a variety of methods of diagnosing a bone-related disorder in a human patient. In one embodiment of such a method, the nucleotide sequence of a portion of an mrr gene of the patient is determined and compared with the nucleotide sequence of the corresponding portion of the mrr gene of a human not afflicted with the disorder, or with the corresponding portion of a normal mrr gene, such as the mrr gene which occurs in the cosmid(s) corresponding to GenBank Accession No. AF003625. A difference between the nucleotide sequence of the portion of the gene of the patient and the corresponding portion of the normal mrr gene (or the corresponding portion of GenBank Accession No. AF003625, preferably including at least nucleotide residues 27599 through 32411, see FIG. 5) is an indication that the patient is afflicted with the bone-related disorder.

In another embodiment of a method of diagnosing a bone-related disorder in a human patient, the nucleotide sequence of a portion of a transcript polynucleotide of a tissue of the patient is determined. A “transcript polynucleotide” is a pre-mRNA (i.e. a transcription product that can, optionally, be partially spliced), an mRNA (i.e. a mature, fully-spliced gene transcript), a cDNA prepared by reverse transcription of a pre-mRNA or an mRNA, or a portion of one of these. The transcript polynucleotide corresponds to an mrr gene of the patient. The sequence of the transcript polynucleotide is compared with the nucleotide sequence of the corresponding portion of a normal mrr gene transcript (e.g. normal human mrr cDNA, as in SEQ ID NO: 2). A difference between the two sequences is an indication that the patient is afflicted with the bone-related disorder;

Another embodiment of the method of diagnosing a bone-related disorder in a human patient involves determining the amino acid sequence of a portion of an MRR protein of a tissue of the patient. This sequence is compared with the amino acid sequence of the corresponding portion of a normal MRR protein (e.g. an MRR protein produced by bone cells of a human not afflicted with a bone-related disorder, as in SEQ ID NO: 1). A difference between the amino acid sequence of the portion of the patient's MRR protein and the amino acid sequence of the corresponding portion of the normal MRR protein (e.g. the corresponding portion of SEQ ID NO: 1) is an indication that the patient is afflicted with the bone-related disorder. The portion of the MRR protein for which the sequence is determined according to this method is preferably the entirety of the MRR protein (i.e. the portion corresponding to all of SEQ ID NO: 1).

In yet another embodiment of the method of diagnosing a bone-related disorder in a human patient, the level of expression of an mrr gene in a tissue of the patient is determined and compared with the level of expression of the mrr gene in the same tissue of a human not afflicted with the bone-related disorder. A difference between the level of expression of the mrr gene in the tissue of the patient and the level of expression of the mrr gene in the same tissue of the human not afflicted with the bone-related disorder is an indication that the patient is afflicted with the bone-related disorder;

A bone-related disorder can also be diagnosed in a human patient by determining the MRR protein content of a tissue of the patient and comparing that protein content and the MRR protein content of the same tissue of a human not afflicted with the bone-related disorder. A difference between the two protein content values is an indication that the patient is afflicted with the bone-related disorder.

In still another method of diagnosing a bone-related disorder in a human patient, a detectable MRR-binding agent to the patient and the amount of the MRR-binding agent in the patient (i.e. either the total amount in the body of the patient or the total amount associated with a bone tissue of the patient) is detected. This amount is compared with the corresponding (i.e. whole-body or bone tissue-associated) amount of the MRR-binding agent that is detected in a human who is not afflicted with the bone-related disorder and to whom the MRR-binding agent has been administered. A difference between the amount of the MRR-binding agent detected in the patient and the amount of the MRR-binding agent detectable in the human not afflicted with the bone-related disorder following administration of the MRR-binding agent thereto is an indication that the patient is afflicted with the bone-related disorder.

The MRR-binding agent used in this method can be substantially any detectable agent which binds specifically (i.e. to a greater degree than with any other protein naturally associated with bone tissue) with MRR protein, particularly human MRR protein. Representative MRR-binding agents include labeled MRR ligands and labeled antibody substances which bind specifically with MRR. When the MRR-binding agent is an antibody substance, for example, the label may be any composition that can be attached to or substantially irreversibly associated with the antibody substance and that can be detected by one or more (preferably extracorporeal) detection methods. Examples of labels include heavy metal atoms, radio-opaque compounds, and compounds (e.g. spin-labeled compounds) that can be detected by nuclear magnetic resonance imaging. Preferably, the label is one which can be quantitated using an extracorporeal method.

In each of the methods of diagnosing a bone-related disorder described in this section, the patient tissue used in the method can be a tissue that is located in the patient, or it can be a tissue that is removed from the patient (i.e. a tissue sample). The tissue is preferably a bone tissue, but other tissues may be used, particularly when the genomic nucleotide sequence is determined in the assay method. When a tissue sample is used in one of these diagnostic methods, the sample is preferably a bone tissue biopsy, a blood fluid, or a cerebrospinal fluid. Blood fluid (e.g. whole blood, lymph, or partially or homogeneously purified components of blood) and cerebrospinal fluid (e.g. fluid withdrawn from one or both of the epidural and subarachnoid spaces) samples are preferred over bone tissue biopsy samples, owing to the relatively greater invasiveness of the procedures necessary to obtain bone tissue samples.

It is not necessary that two human tissues or tissue samples be used for each diagnostic assay. Although a tissue or tissue sample from the patient being diagnosed is indispensable, the tissue or tissue sample of the human not afflicted with the bone-related disorder is not always required (i.e. the ‘reference sample’). As a substitute, the reference sample may be replaced either with pooled tissue samples collected from a plurality of humans not afflicted with the bone-related disorder or with a consensus profile (i.e. data rather than tissue) of assay results generated using a plurality of tissue samples obtained from humans not afflicted with the disorder. It is anticipated that as the diagnostic methods of the invention are used more regularly, this consensus profile will be based on an increasingly large number of ‘normal’ samples and will eventually eliminate any need for further diagnostic testing of humans known not to be afflicted with the relevant disorder.

Prognostic Methods for Bone-Related Disorders

The methods described in the previous section are useful for determining whether a human patient is presently afflicted with a bone-related disorder. To some extent, the diagnostic methods are useful for detecting bone-related disorders at a very early stage, i.e. prior to manifestation of symptoms of the disorder that are discernable to the patient. To the extent that such very early diagnoses are possible, the ‘diagnostic’ methods of the invention may be used as prognostic methods for predicting whether a patient will become afflicted with a bone-related disorder. However, the invention also includes prognostic methods which, in certain circumstances, permit detection of a predisposition of a patient to become afflicted with a bone-related disorder even when the diagnostic methods of the invention cannot be used to detect an abnormality.

One such method involves determining the nucleotide sequence of a portion of an mrr gene of the patient and comparing this sequence and the corresponding portion of an mrr gene of a human who is not afflicted with and does not, will not, or did not (depending on the sample used) become afflicted with the disorder during his or her lifetime. For example, the mrr gene of the non-afflicted human can have the nucleotide sequence listed in GenBank Accession No. AF003625 (i.e. including at least nucleotide residues 27599 through 32411 thereof). A difference between the nucleotide sequences of the portion of the mrr gene of the patient and the corresponding portion of the non-afflicted human's mrr gene (or the corresponding portion of GenBank Accession No. AF003625) is an indication that the patient is predisposed to become afflicted with the bone-related disorder.

In another embodiment of a prognostic method of the invention, the nucleotide sequence is determined for a portion of a transcript polynucleotide (i.e. as described in the section relating to the diagnostic methods of the invention) of a tissue of the patient. The transcript polynucleotide corresponds to an mrr gene of the patient. This nucleotide sequence is compared with the corresponding portion of a transcript polynucleotide corresponding to an mrr gene of a human who does (or did) not become afflicted with the bone-related disorder during his or her lifetime (e.g. SEQ ID NO: 2). A difference between the two nucleotide sequences is an indication that the patient is predisposed to become afflicted with the bone-related disorder.

Another prognostic method of the invention involves determining the amino acid sequence of a portion of an MRR protein of a tissue of the patient. This sequence is compared with the corresponding portion of the amino acid sequence of an MRR protein of a human who does (or did) not become afflicted with the bone-related disorder during his or her lifetime (e.g. SEQ ID NO: 1). A difference between these two amino acid sequences is an indication that the patient is predisposed to become afflicted with the bone-related disorder.

As with the diagnostic methods of the invention, use of a bone tissue of the patient is preferred in order to obtain results which are the most clinically relevant to the affected (i.e. bone) tissue. However, use of blood fluids and cerebrospinal fluids are also preferred, owing to the convenience with which they may be collected.

Methods of Treating a Bone-Related Disorder

The invention also includes several methods of treating or alleviating a bone-related disorder in a human patient (i.e. reducing the severity of at least one symptom of the disorder or the frequency with which at least one symptom of the disorder is experienced by the patient). It is recognized that there are at least two general classes of bone-related disorders, including those which are characterized by general or localized decrease in bone density and those which are characterized by general or localized increase in bone density. The ordinarily skilled artisan is able, through the exercise of no more than ordinary, if any, experimentation, to determine which of the therapeutic methods described herein are appropriate for a particular patient, taking into account such factors as the nature and stage of progression of the bone-related disorder, the age, gender, diet, and general state of health of the patient, and other relevant factors within the ken of the ordinarily skilled artisan.

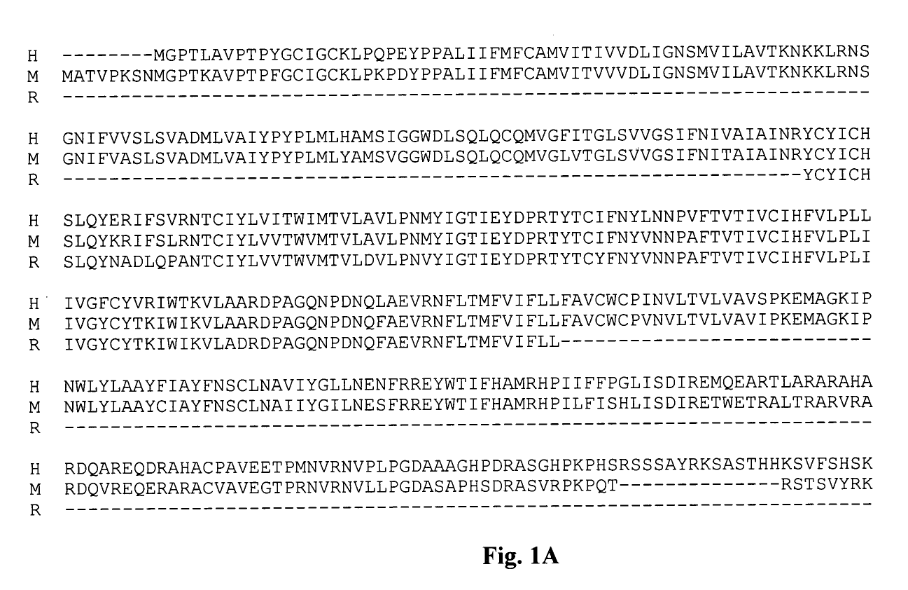

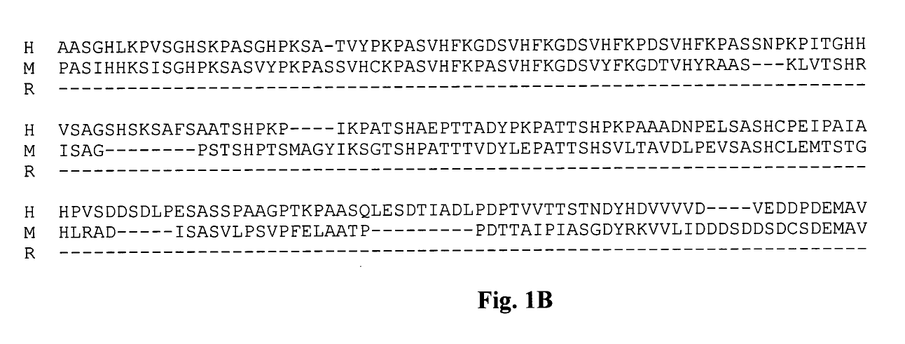

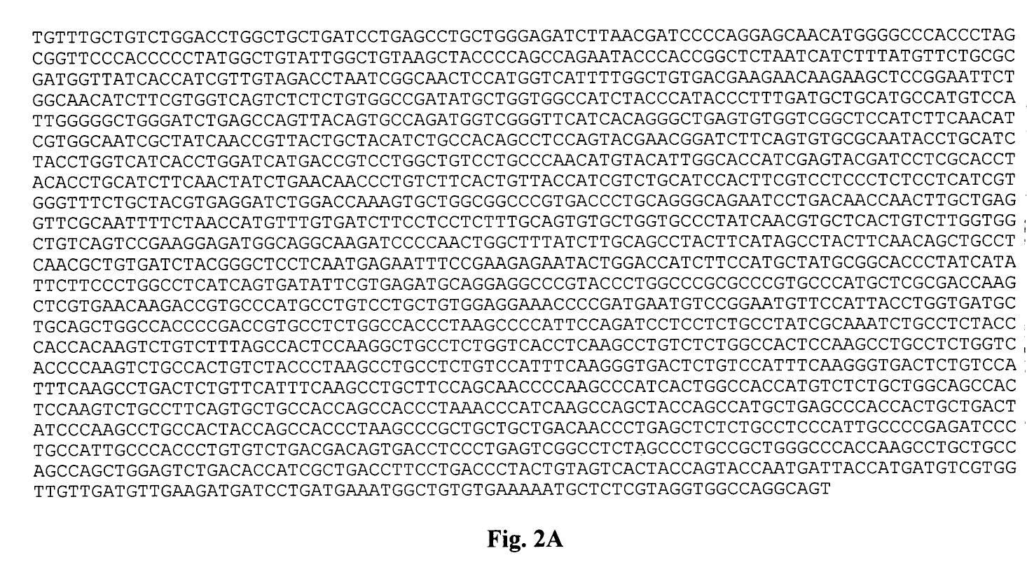

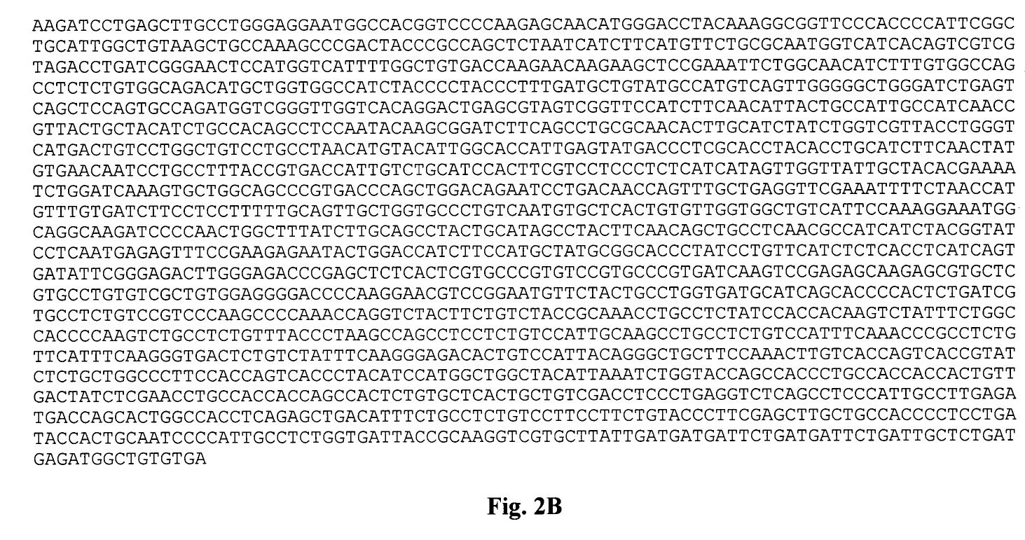

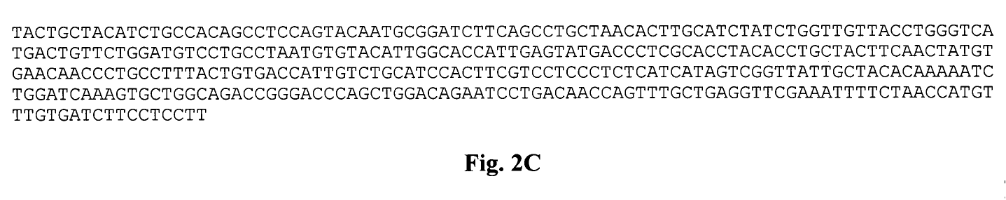

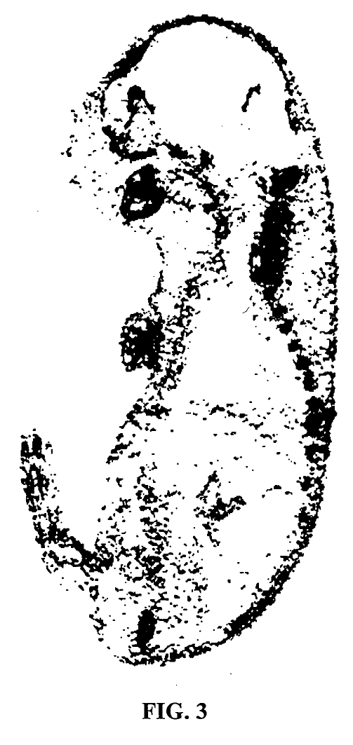

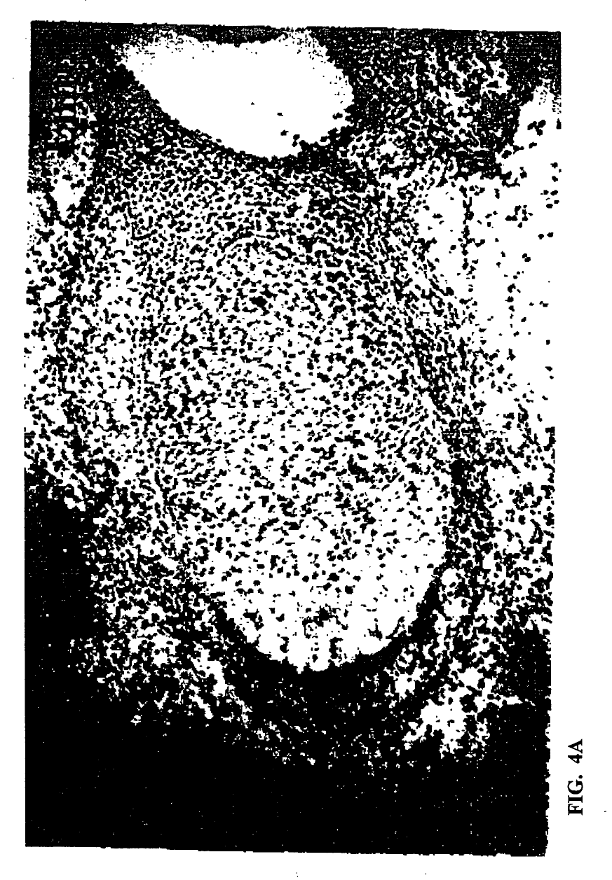

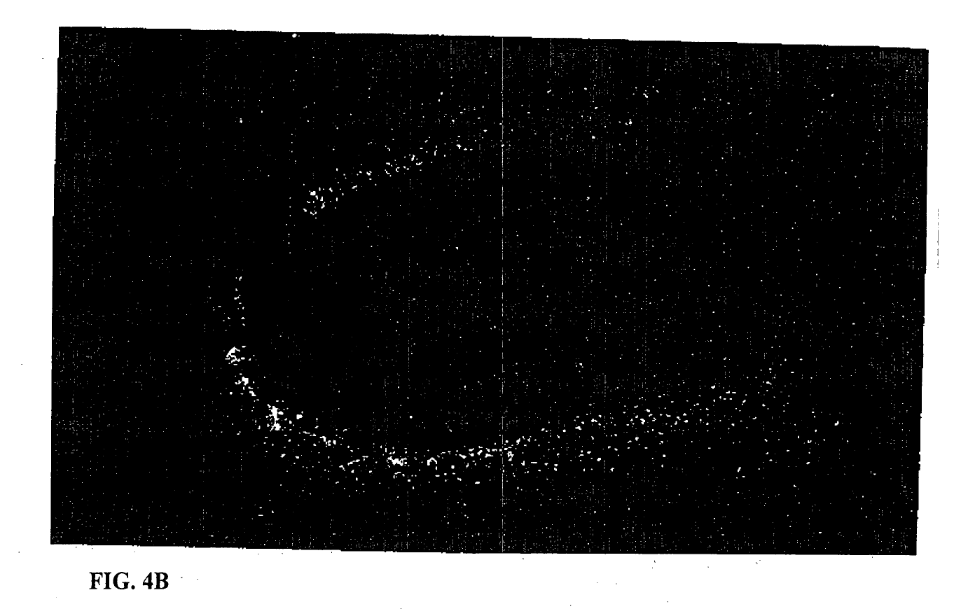

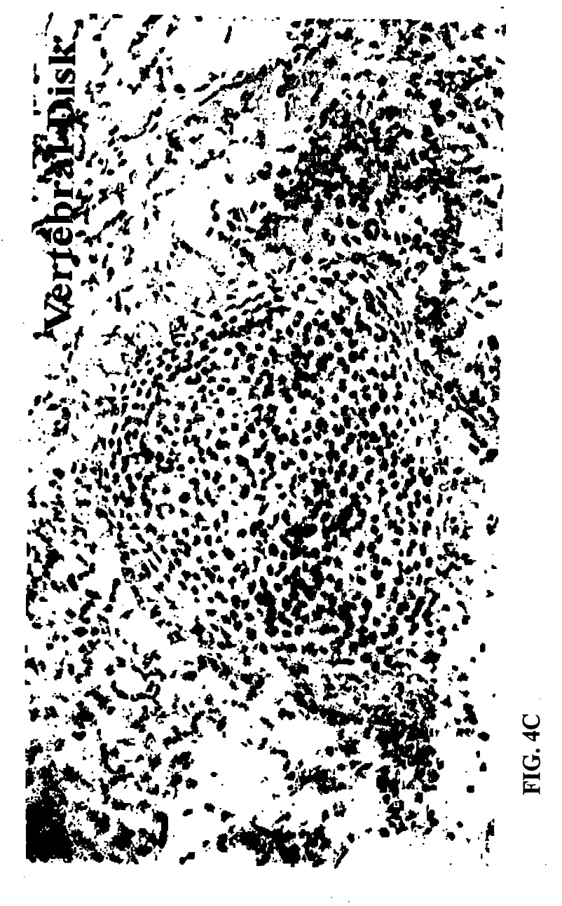

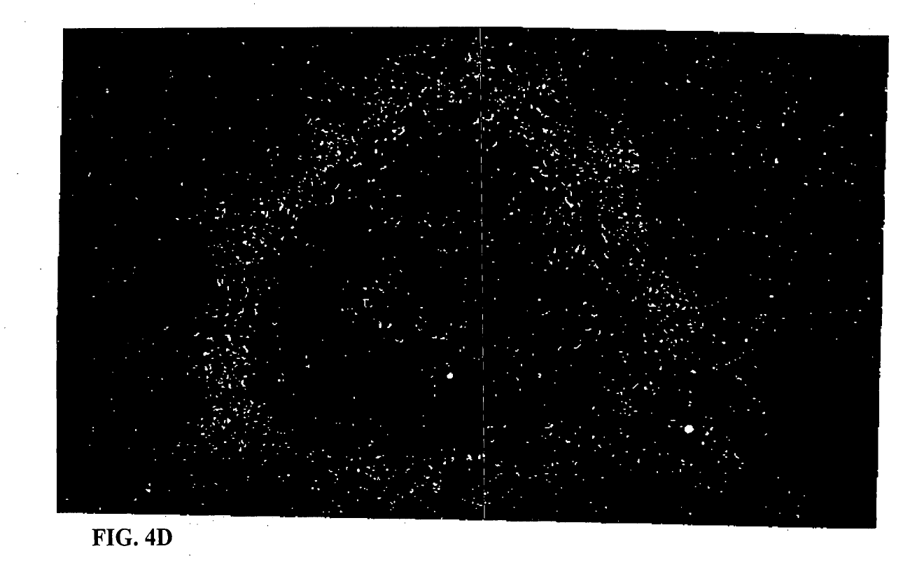

One therapeutic method of the invention involves providing a biologically active portion of an MRR protein to a bone tissue of the patient in an amount sufficient to alleviate the bone-related disorder. This method is useful when the disorder is characterized by insufficient or absent MRR activity in a bone tissue in which MRR activity is normally greater. Preferably, the biologically active portion of the MRR protein is a protein having the amino acid sequence SEQ ID NO: 1 (see FIG. 1). The MRR protein may be provided to the bone tissue by administering (e.g. topically during a surgical procedure or orally) the protein to the tissue. Alternatively, the biologically active portion of the MRR protein can be provided to the bone tissue by providing an expression vector comprising a polynucleotide encoding the biologically active portion of the MRR protein (e.g. a polynucleotide having the sequence SEQ ID NO: 2).

In another embodiment of the therapeutic methods of the invention, a non-functional MRR protein is provided to a bone tissue of the patient in an amount sufficient to alleviate the bone-related disorder. A non-functional MRR protein is a protein which exhibits at least one biological activity which is characteristic of the biologically active MRR protein (e.g. ligand binding or ligand-binding-dependent enzymatic activity), but which also fails to exhibit at least one other biological activity which is characteristic of the biologically active MRR protein. Alternatively, the non-functional MRR protein can be provided to the bone tissue by providing an expression vector comprising a polynucleotide encoding the non-functional MRR protein to the bone tissue of the patient in an amount sufficient to alleviate the bone-related disorder. This therapeutic method is useful for bone-related disorders which are characterized by a greater-than-normal level of MRR protein activity in the bone tissue, a greater-than-normal level of the ligand of MRR protein in an extracellular fluid which contacts the bone tissue (e.g. a blood fluid or a cerebrospinal fluid), or both.

Another therapeutic method of the invention involves providing an antagonist of MRR protein activity (e.g. an antibody substance which binds specifically with MRR protein) to a bone tissue of the patient in an amount sufficient to alleviate the bone-related disorder. This method is useful for treatment of bone-related disorders characterized by over-production of MRR protein in the bone tissue (i.e. relative to MRR protein expression in humans not afflicted with the disorder), by the presence of abnormally high levels of the ligand of MRR protein in an extracellular fluid which contacts the bone tissue, or by both. The antagonist may, for example, be an antagonist identified by using a method described herein for identifying a composition which modulates an activity of MRR protein. Another method of the invention for alleviating disorders of this sort involves providing an inhibitor of mrr expression to the bone tissue of the patient in an amount sufficient to alleviate the disorder. Examples of inhibitors include anti-sense oligonucleotides and ribozymes for inhibiting expression of the mrr gene, as described elsewhere herein.

Bone-related disorders which are characterized by abnormally low production of MRR protein in a bone tissue of the patient, by the presence of abnormally low levels of the ligand of MRR protein in an extracellular fluid which contacts the bone tissue, or by both can be treated, for example, by providing an agonist of MRR protein activity to the bone tissue in an amount sufficient to alleviate the bone-related disorder. Alternatively, an enhancer of mrr expression can be provided to the bone tissue of the patient in an amount sufficient to alleviate the bone-related disorder by enhancing mrr expression.

Pharmaceutical compositions, formulations, dosing information, and other relevant pharmacological information relevant to provision of one or more agents described in these therapeutic methods are described elsewhere herein.

Prevention/Inhibition of a Bone-Related Disorder

The invention also includes methods of preventing or inhibiting a bone-related disorder in a human patient at risk for developing the disorder (i.e. delaying the onset of one or more symptoms or diminishing the severity of one or more symptoms of the disorder upon the onset of the disorder). Patients can be determined to be ‘at risk for developing’ a bone-related disorder, for example, by considering the age of the patient, the patient's family history relating to the disorder, risk factors recognized for the particular disorder, and other factors which will be understood by the ordinarily skilled artisan. The artisan is also able to select from the preventive/inhibitory methods of the invention based, among other factors, on the identity of the bone-related disorder to be prevented or inhibited, the risk that the patient will develop the disorder, and the age, general health, and family history of the patient.

The invention includes a bone-disorder preventive/inhibitory method which comprises providing a biologically active portion of an MRR protein to a bone tissue of the patient in an amount sufficient to inhibit the bone-related disorder. A method such as this is particularly useful for preventing or inhibiting a bone-related disorder which is characterized by insufficient production of MRR protein in the affected bone tissue. This method is also useful for preventing or inhibiting certain bone-related disorders which are characterized by abnormally low activity of an MRR protein, even though the protein is produced at a normal level. The biologically active portion of the MRR protein can be provided to the bone tissue by providing the protein directly or by providing to the tissue an expression vector comprising a polynucleotide encoding the biologically active portion of the MRR protein.

Another method of preventing or inhibiting a bone-related disorder involves providing a non-functional MRR protein to a bone tissue of the patient in an amount sufficient to inhibit the bone-related disorder. This method is especially useful for prevention and inhibition of disorders characterized by abnormally high levels of MRR production in the affected bone tissue and disorders characterized by normal levels of MRR protein which exhibits abnormally high activity. The non-functional MRR protein can be provided to the bone tissue by providing the non-functional protein directly or by providing to the tissue an expression vector comprising a polynucleotide encoding the non-functional MRR protein.

Bone-related disorders characterized by abnormally high levels of MRR protein or by abnormally high MRR protein activity can also be inhibited or prevented by providing an antagonist of MRR protein activity or an inhibitor of mrr gene expression to a bone tissue of the patient in an amount sufficient to inhibit the bone-related disorder. Examples of antagonists include antibody substances which bind specifically with MRR protein and MRR protein ligand analogs. Such antagonists can be identified, for example, using a screening method described herein for identifying a composition useful for alleviating the disorder. Inhibitors of mrr gene expression include, for example, anti-sense oligonucleotides and ribozymes, as described herein.

Bone-related disorders characterized by abnormally low levels of MRR protein or by abnormally low MRR protein activity can be inhibited or prevented by providing an agonist of MRR protein activity or an enhancer of mrr gene expression to a bone tissue of the patient in an amount sufficient to inhibit the bone-related disorder. Such agonists and enhancers can be identified, for example, using a screening method described herein for identifying a composition useful for alleviating the disorder.

Screening Methods for Identifying Bone-Related Disorder Therapeutic Agents

The invention provides a method (also referred to herein as a “screening assay”) for identifying modulators, i.e., candidate or test compounds or agents (e.g., peptides, peptidomimetics, small molecules or other drugs) which bind with a vertebrate (e.g. human) MRR protein of the invention, or have a stimulatory or inhibitory effect on, for example, expression or activity of an MRR protein of the invention.

In one embodiment, the invention provides assays for screening candidate or test compositions which bind with or modulate the activity of the membrane-bound form of an MRR protein of the invention, or a biologically active portion thereof. The test compositions of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is generally limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869), or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

Another set of candidate compounds include full-length MRR or soluble fragments thereof that competes for ligand binding. Other candidate compounds include mutant MRR proteins and fragments containing mutations that affect MRR protein function and competitively inhibit ligand binding with non-mutant MRR.

In one embodiment, an assay is a cell-based assay in which a cell which expresses a membrane-bound form of an MRR protein of the invention, or a biologically active portion thereof, on the cell surface is contacted with a test compound and the ability of the test compound to bind with the polypeptide is determined. The cell, for example, can be a yeast cell or a cell of vertebrate or mammalian origin. Determining the ability of the test compound to bind with the MRR protein can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In yet another embodiment, an assay of the present invention is a cell-free assay comprising contacting an MRR protein of the invention, or a biologically active portion thereof, with a test compound and determining the ability of the test compound to bind with the polypeptide or biologically active portion thereof. Binding of the test compound to the polypeptide can be determined either directly or indirectly as described above. In a preferred embodiment, the assay includes contacting the MRR protein of the invention, or a biologically active portion thereof, with a known compound which binds the MRR protein to form an assay mixture, contacting the assay mixture with a test compound, and determining the ability of the test compound to interact with the MRR protein, wherein determining the ability of the test compound to interact with the MRR protein comprises determining the ability of the test compound to preferentially bind with the MRR protein or biologically active portion thereof as compared to the known compound.

The invention encompasses several different methods for determining whether a test composition is useful for alleviating a bone-related disorder. For example, in one method, a cell which comprises a functional MRR protein is maintained in the presence of the test composition. An activity of the MRR protein of the cell maintained in the presence of the test composition is compared with the same activity of the MRR protein of a cell of the same type maintained in the absence of the test composition. A difference between an activity of the MRR protein of the cell maintained in the presence of the test composition and the same activity of the MRR protein of the cell of the same type maintained in the absence of the test composition is an indication that the test composition is useful for alleviating a bone-related disorder. The biologically active MRR protein can, for example, be a protein having an amino acid sequence selected from the group consisting of SEQ ID NOs: 1 and 3. Preferably, the MRR protein is a human MRR protein (e.g. a protein having the amino acid sequence SEQ ID NO: 1).

In another embodiment of the screening methods of the invention, a cell which comprises a functional MRR protein is maintained in the presence of the test composition. The level of expression of the mrr gene in the cell maintained in the presence of the test composition is compared with the level of expression of the mrr gene in the MRR protein of a cell of the same type maintained in the absence of the test composition. A difference between the two levels of expression is an indication that the test composition is useful for alleviating a bone-related disorder.

In yet another embodiment of these screening methods, a cell which comprises a functional MRR protein is maintained in the presence of the test composition. A bone phenotype of the cell maintained in the presence of the test composition is compared with the same bone phenotype of a cell of the same type maintained in the absence of the test composition. A difference between the bone phenotype of the two cells is an indication that the test composition is useful for alleviating a bone-related disorder. The bone phenotype may, for example be a bone deposition phenotype (e.g. a phenotype characterized by abnormally high production of bone organic elements by the cell or by an abnormally high degree of mineralization in the presence of the cell), a bone resorption phenotype (e.g. a phenotype characterized by abnormally low production of bone organic elements by the cell or by an abnormally high degree of bone mineral dissolution in the presence of the cell), or a bone morphology phenotype (e.g. a phenotype characterized by an abnormal organic/mineral element composition or arrangement in bone associated with the cell).

Another embodiment of the screening methods of the invention for determining whether a test composition is useful for alleviating a bone-related disorder involves administering the test composition to a first animal which naturally harbors an mrr gene. A bone phenotype of the first transgenic animal is compared with the same bone phenotype of a second animal which naturally harbors an mrr gene and to which the test composition is not administered. A difference between the two phenotypes is an indication that the test composition is useful for alleviating a bone-related disorder. Transgenic animals useful in this embodiment may be made as described elsewhere herein.

Still another method of determining whether a test composition is useful for alleviating a bone-related disorder involves administering the test composition to a first non-human transgenic animal which harbors an exogenous mrr gene. A bone phenotype of the first transgenic animal is compared with the same bone phenotype of a second non-human transgenic animal which harbors an exogenous mrr gene and to which the test composition is not administered. A difference between the bone phenotypes of the two transgenic animals is an indication that the test composition is useful for alleviating a bone-related disorder.

The invention also includes a method of determining whether a test composition is useful for alleviating a bone-related disorder, wherein an artificial membrane which comprises a biologically active MRR protein is maintained in the presence of the test composition. An activity of the MRR protein of the artificial membrane maintained in the presence of the test composition is compared with the same activity of the MRR protein of an artificial membrane of the same type maintained in the absence of the test composition. A difference between the activities of two proteins is an indication that the test composition is useful for alleviating a bone-related disorder. An artificial membrane is a lipid bilayer into which an MRR protein can be inserted without complete loss of its activity. Examples of artificial membranes include liposomes and re-sealed erythrocytes.

In screening methods of the invention that involve determining the activity of an MRR protein, the activity may, for example, be selected from the group consisting of a proteolytic activity (e.g. ability of the carboxyl terminal portion of MRR protein to cleave a peptide bond of a polypeptide), a pore-modulating activity (e.g. ability of MRR protein to activate or de-activate a transmembrane protein pore), an enzyme-modulating activity (e.g. ability of MRR protein to modify an enzyme in a manner that increases or decreases the enzyme's activity), and a gene transcription-modulating activity (e.g. ability of MRR protein to enhance or inhibit expression of a gene).

Interactions between MRR protein and test compositions can also be assessed using chimeric receptor proteins in which the amino terminal extracellular domain, or parts thereof, the entire transmembrane domain or sub-regions, such as any of the seven transmembrane segments or any of the intracellular or extracellular loops and the carboxyl terminal intracellular domain, or parts thereof, are be replaced by heterologous domains or sub-regions. For example, a G-protein-binding region can be used that interacts with a different G-protein than that which is recognized by the native MRR protein. Accordingly, a different set of signal transduction components is available as an end-point assay for activation. Alternatively, the entire transmembrane portion or sub-regions (e.g. the transmembrane segments or intracellular or extracellular loops) can be replaced with the entire transmembrane portion or sub-regions specific to a host cell that is different from the host cell from which the amino terminal extracellular domain and/or the G-protein-binding region are derived. This allows assays to be performed in other than the specific host cell from which the receptor is derived. Alternatively, the amino terminal extracellular domain (or other ligand-binding regions, or both) can be replaced by a domain (or other binding region, or both) which binds a different ligand, thereby providing an assay for test compounds that interact with the heterologous amino terminal extracellular domain (or region) but still cause signal transduction. Furthermore, activation can be detected using a reporter gene containing an easily detectable coding region operably linked to a transcriptional regulatory sequence that is part of the native signal transduction pathway.

In screening methods that involve cells, the cell is preferably an animal cell, and more preferably a bone cell. For example, the cell can be a human cell, a mouse cell, or a rat cell, although other (e.g. yeast or nematode) cells may also be used in the screening methods. Likewise, substantially any animal mrr gene can be used. However, vertebrate mrr genes are preferred. For example, the gene may be a human mrr gene, a mouse mrr gene, or a rat mrr gene, such as one of those described in the prior art. Preferably, the mrr gene encodes a protein having an amino acid sequence selected from the group consisting of SEQ ID NOs: 1 and 3.

In one or more embodiments of the above screening assay methods of the present invention, it can be desirable to immobilize either an MRR protein of the invention or a ligand thereof in order to facilitate separation of complexed from non-complexed forms of one or both of these species, as well as to accommodate automation of the assay. Binding of a test composition to the polypeptide, or interaction of the polypeptide with a ligand in the presence and absence of a test composition, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione Sepharose beads (Sigma Chemical; St. Louis, Mo.) or glutathione derivatized microtiter plates, which are then combined with the test compound or the test compound and either the non-adsorbed ligand or an MRR protein of the invention, and the mixture incubated under conditions conducive to complex formation (e.g., at physiological conditions for salt and pH). Following incubation, the beads or microtiter plate wells are washed to remove any non-bound components and complex formation is measured either directly or indirectly, for example, as described above. Alternatively, the complexes can be dissociated from the matrix, and the level of binding or activity of the MRR protein of the invention can be determined using standard techniques.

Other techniques for immobilizing proteins on matrices can also be used in the screening assays of the invention. For example, an MRR protein of the invention or a ligand thereof can be immobilized utilizing conjugation of biotin and streptavidin. Biotinylated polypeptides or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with MRR protein or a ligand thereof but which do not interfere with binding of MRR protein to its ligand can be derivatized to the wells of the plate, and non-bound ligand or MRR protein of the invention trapped in the wells by antibody conjugation. Methods for detecting such complexes, in addition to those described above for the GST-immobilized complexes, include immunodetection of complexes using antibodies reactive with MRR protein or a ligand thereof, as well as enzyme-linked assays which rely on detecting an enzymatic activity associated with MRR protein or a ligand thereof. It can be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl) dimethylamminio]-1-propane sulfonate (CHAPS), 3-[(3-cholamidopropyl) dimethylamminio]-2-hydroxy-1-propane sulfonate (CHAPSO), or N-dodecyl-N,N-dimethyl-3-ammonio-1-propane sulfonate.

In another embodiment, modulators of expression of an MRR protein of the invention are identified in a method in which a cell is contacted with a test composition and expression of the selected mRNA or protein (i.e., the mRNA or protein corresponding to a polypeptide or nucleic acid of the invention) in the cell is determined. The level of expression of the selected mRNA or protein in the presence of the test composition is compared to the level of expression of the selected mRNA or protein in the absence of the test composition. The test composition can then be identified as a modulator of expression of MRR protein based on this comparison. For example, when expression of the selected mRNA or protein is greater (i.e. statistically significantly greater) in the presence of the test composition than in its absence, the test composition is identified as a stimulator of the selected mRNA or protein expression. Alternatively, when expression of the selected mRNA or protein is less (i.e. statistically significantly less) in the presence of the test composition than in its absence, the test composition is identified as an inhibitor of the selected mRNA or protein expression. The level of the selected mRNA or protein expression in the cells can be determined by methods described herein.

In yet another aspect of the invention, an MRR protein of the invention can be used as a “bait protein” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind with or interact with MRR protein and modulate its activity. Such binding proteins are also likely to be involved in the propagation of signals by MRR protein as, for example, upstream or downstream elements of a signaling pathway involving MRR protein.

This invention further pertains to novel agents identified by the above-described screening assays and uses thereof for treatments as described herein.

The propensity of compounds to induce one or more bone-related disorders has undergone only limited testing, owing in part to the difficulty of determining such a propensity. The invention includes a method of determining the propensity of a test compound to induce a bone-related disorder in a human patient. This screening assay is performed in the same way as any of the screening assays for determining whether a test compound is useful for alleviating a bone-related disorder. However, instead of attempting to identify compounds that reduce the aberrant effects attributable to the bone-related disorder, one attempts to identify compounds that induce or enhance such aberrant effects.

mrr Polymorphisms Associated with Bone-Related Disorders

The invention also includes a method of identifying a polymorphism (e.g. a single nucleotide polymorphism, a deletion, a rearrangement, etc.) associated with an mrr gene of a human patient afflicted with a bone-related disorder. This method comprises determining the nucleotide sequence of a polynucleotide associated with the mrr gene in the patient. This nucleotide sequence is compared with the nucleotide sequence of the corresponding portion of an mrr gene of a human not afflicted with the bone-related disorder. A difference between the two sequences indicates a polymorphism associated with the mrr gene of the human patient afflicted with the bone-related disorder. In this method, it is not necessary that the entire sequence of each of the two polynucleotides be determined. Indeed, it is preferred that any of a wide variety of well known methods be used to identify regions of non-identical sequence between the two polynucleotides.

Individuals carrying polymorphisms in the mrr gene can be detected at the nucleic acid level by a variety of techniques. In certain embodiments, detection of the polymorphism involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting single nucleotide polymorphisms in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. PCR and/or LCR can be desirable to use as a preliminary amplification step in conjunction with any of the techniques used for detecting polymorphisms described herein.

Alternative amplification methods include: self-sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, polymorphisms in a selected gene from a sample cell can be identified by alterations in restriction enzyme cleavage patterns. For example, sample and control DNA is isolated, (optionally) amplified, digested with one or more restriction endonucleases, and fragment length sizes are determined by gel electrophoresis and compared. Differences in fragment length sizes between sample and control DNA indicates one or more polymorphisms in the sample DNA. Moreover, the use of sequence specific ribozymes (see, e.g., U.S. Pat. No. 5,498,531) can be used to score for the presence of specific polymorphisms by development or loss of a ribozyme cleavage site.

In other embodiments, polymorphisms can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, polymorphisms can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of single nucleotide polymorphisms. This step is followed by a second hybridization array that allows the characterization of specific polymorphisms by using smaller, specialized probe arrays complementary to all polymorphisms detected. Each array is composed of parallel probe sets, one complementary to the wild-type gene and the other complementary to the mutant gene.

In yet another embodiment, any of a variety of sequencing reactions known in the art can be used to directly sequence the selected gene and detect polymorphisms by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting polymorphisms in a selected gene include methods in which protection from cleavage agents is used to detect mismatched bases in RNA/RNA or RNA/DNA heteroduplexes (Myers et al. (1985) Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. RNA/DNA duplexes can be treated with RNase to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of polymorphism(s). See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called DNA mismatch repair enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to one embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify polymorphisms in genes. For example, single strand conformation polymorphism (SSCP) can be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids are denatured and allowed to re-nature. The secondary structure of single-stranded nucleic acids varies according to sequence, and the resulting alteration in electrophoretic mobility enables the detection of even a single nucleotide polymorphism. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of polymorphic fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a ‘GC clamp’ of approximately 40 base pairs of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting single nucleotide polymorphisms include, but are not limited to, selective oligonucleotide hybridization, selective amplification, or selective primer extension. For example, oligonucleotide primers can be prepared in which the known polymorphism is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different polymorphisms when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification can carry the polymorphism of interest in the center of the molecule (so that amplification depends on differential hybridization; Gibbs et al. (1989) Nucleic Acids Res. 17:2437-2448) or at the extreme 3′end of one primer where, under appropriate conditions, mismatching can prevent or reduce polymerase extension (Prossner (1993) Tibtech 11:238). In addition, it can be desirable to introduce a novel restriction site in the region of the mutation to create cleavage-based detection (Gasparini et al. (1992) Mol. Cell Probes 6:1). Amplification can also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, using pre-packaged diagnostic kits comprising at least one probe nucleic acid or antibody reagent described herein, which can be conveniently used, e.g., in clinical settings to diagnose patients exhibiting symptoms or family history of a disease or illness involving a gene encoding a polypeptide of the invention. Furthermore, any cell type or tissue (e.g. a neuronal tissue) in which a polypeptide of the invention is expressed can be utilized in the prognostic assays described herein.

Because, as described herein, MRR is a GPCR, MRR can also exhibit one or more properties which GPCRs commonly exhibit. For example, GPCRs are known to participate in transmembrane signaling systems. In such systems, the GPCR acts as an integral membrane transducer which couples binding of a ligand to the GPCR on the extracellular side of the membrane to modulation of the physiological activity of one or more proteins within the cell cytoplasm. Most GPCRs directly modulate one or more physiological activities of a G-protein which is capable of associating with the intracellular (i.e. carboxyl terminal) portion of the GPCR. The corresponding G-protein, the GPCR, or both, also can exhibit one or more of the following activities:

a proteolytic activity (e.g. ability to cleave an aminoacyl bond of a polypeptide),

a pore-modulating activity (e.g. ability to ‘open’ a transmembrane protein pore, that is, to render the transmembrane protein capable of facilitating passage of a compound from one side of the cytoplasmic membrane to the other),

an enzyme-modulating activity (e.g. ability to activate of a nucleotide cyclase enzyme), and

a gene transcription-modulating activity (e.g. ability to enhance transcription of a gene).

Transmembrane signal conduction facilitated by GPCRs enables rapid and specific physiological response to molecules (e.g. endocrine factors such as hormones) present in the extracellular environment without the need for specialized cellular components for transporting the molecule to the interior of the cell. Examples of such physiological responses include interconversion of intracellular molecules that participate in a signal transduction pathway (e.g., phosphatidylinositol 4,5-bisphosphate, inositol 1,4,5-triphosphate, and cyclic AMP), polarization of the cytoplasmic membrane; production or secretion of one or more molecules (e.g. proteins, neurotransmitters, hormones, and the like) by the cell, alteration in the structure of a cellular component, cell proliferation (e.g. synthesis of DNA), cell migration, cell differentiation, and modulation of cell survival (e.g. induction of apoptosis).

Upon binding a ligand at its extracellular (i.e. amino terminal) portion, MRR protein facilitates interconversions of one or more molecules (e.g. proteins, phosphorylated organic compounds, or both) involved in turnover and metabolism of phosphatidylinositol 4,5-bisphosphate (PIP₂). PIP₂ is a phospholipid which occurs in the cytosolic leaflet of the plasma membrane. For example, binding of a ligand to MRR can phospholipase C, which catalyzes hydrolysis of PIP₂ to yield 1,2-diacylglycerol (DAG) and inositol 1,4,5-triphosphate (IP₃). Once formed, IP₃ can diffuse to the endoplasmic reticulum surface where it can bind a calcium channel protein which specifically binds with IP₃. IP₃ binding can induce opening of the channel, allowing calcium ions to be released into the cytoplasm. IP₃ can also be phosphorylated by a specific kinase to form inositol 1,3,4,5-tetraphosphate (IP₄), a molecule which can cause calcium entry into the cytoplasm from the extracellular medium. IP₃ and IP₄ can subsequently be hydrolyzed very rapidly to the inactive products inositol 1,4-biphosphate (IP₂) and inositol 1,3,4-triphosphate, respectively. These inactive products can be recycled by the cell to synthesize PIP₂. Hydrolysis of PIP₂ also yields DAG, which remains in the cell membrane where it can activate the enzyme protein kinase C. Protein kinase C is usually found soluble in the cytoplasm of the cell, but upon an increase in the intracellular calcium concentration, this enzyme can move to the plasma membrane where it can be activated by DAG. Activation of protein kinase C can result in various cellular responses, such as phosphorylation of glycogen synthase or phosphorylation of various transcription factors (e.g. NF-kB).

Upon binding a ligand at its extracellular (i.e. amino terminal) portion, MRR protein can also participate is a cAMP signaling pathway. In such a cAMP signaling pathway, binding of a ligand to MRR leads to activation of adenylate cyclase, which catalyzes synthesis of cAMP. Newly synthesized cAMP can activate a cAMP-dependent protein kinase. This activated kinase can phosphorylate a voltage-gated potassium channel protein, or a protein associated with such a channel, inhibiting the ability of the potassium channel to open in the presence of an action potential. Inability of the potassium channel to open results in a decrease in the outward flow of potassium, which can reduce or inhibit polarization of the cytoplasmic membrane.

Thus, MRR protein can participate in one or more of these signaling systems in humans, in other animals in which it naturally occurs, and in cells into which it has been introduced.

Various aspects of the invention are described in further detail in the following subsections.

I. Isolated Nucleic Acid Molecules

One aspect of the invention pertains to isolated nucleic acid molecules that encode a polypeptide of the invention or a biologically active portion thereof (e.g. a portion encoding the seven transmembrane portions of a vertebrate {e.g. human, murine, or rat} MRR protein), as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kilobases, 4 kilobases, 3 kilobases, 2 kilobases, 1 kilobases, 0.5 kilobases or 0.1 kilobases of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of all or a portion of SEQ ID NOs: 2, 4, and 6-9, or a complement thereof, or which has a nucleotide sequence comprising one of these sequences, can be isolated using standard molecular biology techniques and the sequence information provided herein. Using all or a portion of the nucleic acid sequences of one of SEQ ID NOs: 2, 4, and 6-9 as a hybridization probe, nucleic acid molecules of the invention can be isolated using standard hybridization and cloning techniques (e.g., as described in Sambrook et al., Eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of one of SEQ ID NOs: 2, 4, and 6-9, or a portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologs in other cell types, e.g., from other tissues, as well as homologs from other mammals. The probe/primer typically comprises substantially purified oligonucleotide. The oligonucleotide typically comprises a region of nucleotide sequence that hybridizes under stringent conditions to at least about 15, preferably about 25, more preferably about 50, 75, 100, 125, 150, 175, 200, 250, 300, 350, or 400 or more consecutive nucleotides of the sense or anti-sense sequence of one of SEQ ID NOs: 2, 4, and 6-9, or of a naturally occurring mutant of one of SEQ ID NOs: 2, 4, and 6-9.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a biologically active portion of a polypeptide of the invention can be prepared by isolating a portion of one of SEQ ID NOs: 2, 4, and 6, expressing the encoded portion of the polypeptide protein (e.g., by recombinant expression in vitro), and assessing the activity of the encoded portion of the polypeptide.

The invention further encompasses nucleic acid molecules that differ from the nucleotide sequence of one of SEQ ID NOs: 2, 4, and 6-9 due to degeneracy of the genetic code and thus encode the same protein as that encoded by the nucleotide sequence of SEQ ID NOs: 2, 4, or 6.

In addition to the nucleotide sequences of SEQ ID NOs: 2, 4, and 6, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus.

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Two polynucleotides (or two polynucleotide or amino acid sequences) “correspond” to one another if one of the two is highly homologous (i.e. at least 50%, 60%, 70%, 80%, 90%, 95%, or 98% or more homologous) with a polynucleotide (or sequence) present, either consecutively or in an interrupted fashion, within the other. By way of example, both a pre-mRNA transcript of a gene and an mRNA generated by splicing that pre-mRNA correspond to the gene. Further by way of example, the nucleotide sequence AACC corresponds to both the nucleotide sequence AAGGCC and the nucleotide sequence GGAACCGG. The nucleotide sequence AACT also corresponds to both the nucleotide sequence AAGGCC and the nucleotide sequence GGAACCGG, even though it is only 75% homologous to a sequence (AACC) contained within each of these two sequences.

Moreover, nucleic acid molecules encoding proteins of the invention from other species (homologs), which have a nucleotide sequence which differs from that of the rat protein described herein are intended to be within the scope of the invention. Nucleic acid molecules corresponding to natural allelic variants and homologs of a cDNA of the invention can be isolated based on their identity to human nucleic acid molecules using the cDNAs, or a portion thereof, as a hybridization probe according to standard hybridization techniques under stringent hybridization conditions. For example, a cDNA encoding a soluble form of a membrane-bound protein of the invention isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, 5000, 10000, 20000, 40000, or 70000 or more) nucleotides in length and hybridizes under stringent conditions to the nucleic acid molecule comprising the nucleotide sequence, preferably the coding sequence, of one of SEQ ID NOs: 2, 4, and 6-9, or a complement thereof. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of one of SEQ ID NOs: 2, 4, and 6-9, or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention sequence that can exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes which alter amino acid residues that are not essential for activity. Such polypeptides differ in amino acid sequence from one of SEQ ID NOs: 1, 3, and 5, and yet retain biological activity. In one embodiment, the isolated nucleic acid molecule has a nucleotide sequence encoding a protein that includes an amino acid sequence that is at least about 40% identical, 50%, 60%, 70%, 80%, 90%, 95%, or 98% identical to the amino acid sequence of one of SEQ ID NOs: 1, 3, and 5.

An isolated nucleic acid molecule encoding a variant protein can be created by introducing one or more nucleotide substitutions, additions, or deletions into the nucleotide sequence of one of SEQ ID NOs: 2, 4, and 6-9, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), non-charged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein:protein interactions with the polypeptide of the invention; (2) the ability to bind a ligand of the polypeptide of the invention (e.g. another protein identified herein); (3) the ability to bind with a modulator or substrate of the polypeptide of the invention; or (4) the ability to modulate a physiological activity of the protein, such as one of those disclosed herein.

The present invention encompasses antisense nucleic acid molecules, i.e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ non-translated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-₆-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, as described further in the following subsection).

The antisense nucleic acid molecules of the invention are typically administered to a subject or generated in situ such that they hybridize with or bind with cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds with DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind with receptors or antigens expressed on a selected cell surface, e.g., by linking the antisense nucleic acid molecules to peptides or antibodies which bind with cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach (1988) Nature 334:585-591) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved as described in U.S. Pat. No. 4,987,071 and U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety, or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described (Hyrup et al. (1996), supra; Perry-OKeefe et al. (1996) Proc. Natl. Acad. Sci. USA 93: 14670-675).

PNAs can be used in therapeutic and diagnostic applications. For example, PNAs can be used as antisense or anti-gene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a step-wise manner to produce a chimeric molecule with a 5′PNA segment and a 3′DNA segment (Finn et al. (1996) Nucleic Acids Res. 24(17):3357-63). Alternatively, chimeric molecules can be synthesized with a 5′DNA segment and a 3′PNA segment (Peterser et al. (1975) Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

II. Isolated Proteins and Antibodies

One aspect of the invention pertains to isolated proteins, and biologically active portions thereof, as well as polypeptide fragments suitable for use as immunogens to raise antibodies directed against a polypeptide of the invention. In one embodiment, the native polypeptide can be isolated from cells or tissue sources by an appropriate purification scheme using standard protein purification techniques. In another embodiment, polypeptides of the invention are produced by recombinant DNA techniques. As an alternative to recombinant expression, a polypeptide of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide of the invention include polypeptides comprising amino acid sequences sufficiently identical to or derived from the amino acid sequence of the protein (e.g., the amino acid sequence shown in any of SEQ ID NOs: 1, 3, and 5), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence of one of SEQ ID NOs: 1, 3, and 5. Other useful proteins are substantially identical (e.g., at least about 40%, preferably 50%, 60%, 70%, 80%, 90%, 95%, or 99%) to one of SEQ ID NOs: 1, 3, and 5 and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., percent identity is equal to the number of identical positions divided by the total number of positions (e.g., overlapping positions) multiplied by 100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. Id. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

The invention also provides chimeric or fusion proteins. As used herein, a “chimeric protein” or “fusion protein” comprises all or part (preferably a biologically active part) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which the polypeptide of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding a prostaglandin or a thromboxane and inhibiting transmembrane transport thereof. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477). Methods for assessing transmembrane transport of compounds such as prostaglandins and thromboxanes are described elsewhere herein.

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, re-naturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of SEQ ID NOs: 1, 3, and 5, and encompasses an epitope of the protein such that an antibody raised against the peptide forms a specific immune complex with the protein.

Preferred epitopes encompassed by the antigenic peptide are regions that are located on the surface of the protein, e.g., hydrophilic regions. Hydrophobicity plots or similar analyses can be used to identify hydrophilic regions.

An immunogen typically is used to prepare antibodies by immunizing a suitable (i.e. immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention. A molecule which specifically binds with a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition,” as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SURFZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al. (1994) Bio/technology 12:899-903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET ld (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET ld vector relies on transcription from a T7 gn10-lac fusion promoter mediated by a co-expressed viral RNA polymerase (T7 gnl). This viral polymerase is supplied by host strains BL21(DE3) or HMS174(DE3) from a resident λ prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast, or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce non-human transgenic animals. For example, in one embodiment, a host cell of the invention is a fertilized oocyte or an embryonic stem cell into which a sequences encoding a polypeptide of the invention have been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

IV. Pharmaceutical Compositions

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity can, for example, be a small molecule. For example, such small molecules include peptides, peptidomimetics, amino acids, amino acid analogs, polynucleotides, polynucleotide analogs, nucleotides, nucleotide analogs, organic or inorganic compounds (i.e. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Examples of doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram). Examples of doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is about 0.1 mg/kg to 100 mg/kg of body weight (generally about 10 mg/kg to 20 mg/kg). If the antibody is to act in the brain, a dosage of about 50 mg/kg to 100 mg/kg is usually appropriate. Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Additional Uses and Methods of the Invention

Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining expression of a polypeptide or nucleic acid of the invention and/or activity of an MRR protein, in the context of a biological sample (e.g., blood, serum, cells, tissue) to thereby determine whether an individual is afflicted with a disease or disorder, or is at risk of developing a disorder, associated with aberrant expression or activity of an MRR protein. The invention also provides for prognostic (or predictive) assays for determining whether an individual is at risk of developing a disorder associated with aberrant expression or activity of an MRR protein. For example, mutations in a gene encoding MRR protein can be assayed in a biological sample. Such assays can be used for prognostic or predictive purpose to prophylactically treat an individual prior to the onset of a disorder characterized by or associated with aberrant expression or activity of MRR protein.

Another aspect of the invention provides methods for assessing expression of a nucleic acid of the invention, and for assessing activity of MRR protein in an individual to thereby select appropriate therapeutic or prophylactic agents for that individual (referred to herein as “pharmacogenomics”). Pharmacogenomics allows selection of agents (e.g., drugs) for therapeutic or prophylactic treatment of an individual based on the genotype of the individual (e.g., the genotype of the individual is examined to determine the ability of the individual to respond to a particular agent).

Yet another aspect of the invention pertains to monitoring the influence of agents (e.g., drugs or other compounds) on the expression or activity of MRR protein in clinical trials. These and other agents are described in further detail in the following sections.

Diagnostic Assays

An example of a method for detecting the presence or absence of a polypeptide or nucleic acid of the invention in a biological sample involves obtaining a biological sample from a test subject and contacting the biological sample with a compound or an agent capable of detecting the polypeptide or nucleic acid (e.g., mRNA or genomic DNA) such that the presence of the polypeptide or nucleic acid is detected in the biological sample. A preferred agent for detecting mRNA or genomic DNA encoding a polypeptide of the invention is a labeled nucleic acid probe capable of hybridizing to mRNA or genomic DNA encoding the polypeptide. The nucleic acid probe can be, for example, a full-length cDNA, such as the nucleic acid of SEQ ID NOs: 2 and 4 or a portion thereof, such as an oligonucleotide of at least 15, 30, 50, 100, 250 or 500 nucleotides in length and sufficient to specifically hybridize under stringent conditions to a mRNA or genomic DNA encoding MRR protein. Other suitable probes for use in the diagnostic assays of the invention are described herein.

A preferred agent for detecting a polypeptide of the is an antibody capable of binding to the polypeptide, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a polypeptide of the include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a tissue (e.g. a neuronal tissue) sample isolated by conventional means from a subject.

In another embodiment, the methods further involve obtaining a control biological sample from a control subject, contacting the control sample with a compound or agent capable of detecting a polypeptide of the invention or an mRNA or genomic DNA encoding a polypeptide of the invention, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

Pharmacogenomics

Agents, or modulators which have a stimulatory or inhibitory effect on activity or expression of a polypeptide of the invention, as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention, mutation content of a gene of the invention in an individual can be determined to thereby select appropriate agent(s) for therapeutic or prophylactic treatment of the individual.

The field of pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the examples of screening assays described herein.

Monitoring of Effects During Clinical Trials

Monitoring the influence of agents (e.g., drug compounds) on the expression or activity of a polypeptide of the invention can be applied not only in basic drug screening, but also in clinical trials. For example, the effectiveness of an agent, as determined by a screening assay as described herein, to increase gene expression, protein levels, or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity.

For example, and not by way of limitation, genes, including those encoding a polypeptide of the invention, that are modulated in cells by treatment with an agent (e.g., compound, drug or small molecule) which modulates activity or expression of the polypeptide (e.g., as identified in a screening assay described herein) can be identified. Thus, to study the effect of agents on disorders relating to aberrant prostaglandin uptake, for example, in a clinical trial, cells can be isolated and RNA prepared and analyzed for the levels of expression of a gene encoding a polypeptide of the invention or of another gene implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state can be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the pre-administration sample; (iii) obtaining one or more post-administration samples from the subject; (iv) detecting the level the of a polypeptide or nucleic acid of the invention in the post-administration samples; (v) comparing the level of the polypeptide or nucleic acid in the pre-administration sample with the level of the polypeptide or nucleic acid in the post-administration sample or samples; and (vi) altering the administration of the agent to the subject accordingly. For example, increased administration of the agent can be desirable to increase the expression or activity of the polypeptide or nucleic acid to higher levels than detected, i.e., to increase the effectiveness of the agent. Alternatively, decreased administration of the agent can be desirable to decrease expression or activity of the polypeptide or nucleic acid to lower levels than detected, i.e., to decrease the effectiveness of the agent.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of diagnosing a bone-related disorder in a human patient, the method selected from the group consisting of
a) determining the nucleotide sequence of a portion of an mrr gene of the patient and
comparing
i) the nucleotide sequence of the portion and
ii) the corresponding portion of the mrr gene listed in GenBank Accession No. AF003625,
wherein a difference between
i) the nucleotide sequence of the portion and
ii) the corresponding portion of GenBank Accession No. AF003625
is an indication that the patient is afflicted with the bone-related disorder;
b) determining the nucleotide sequence of a portion of a transcript polynucleotide of a tissue of the patient, wherein the transcript polynucleotide corresponds to an mrr gene of the patient and
comparing
i) the nucleotide sequence of the portion and
ii) the corresponding portion of SEQ ID NO: 2,
wherein a difference between
i) the nucleotide sequence of the portion and
ii) the corresponding portion of SEQ ID NO: 2
is an indication that the patient is afflicted with the bone-related disorder;
c) determining the amino acid sequence of a portion of an MRR protein of a tissue of the patient and
comparing
i) the amino acid sequence of the portion of the MRR protein of the tissue of the patient and
ii) the corresponding portion of SEQ ID NO: 1,
wherein a difference between
i) the amino acid sequence of the portion of the MRR protein of the tissue of the patient and
ii) the corresponding portion of SEQ ID NO: 1
is an indication that the patient is afflicted with the bone-related disorder;
d) determining the level of expression of an mrr gene in a tissue of the patient and
comparing
i) the level of expression of the mrr gene in the tissue of the patient and
ii) the level of expression of the mrr gene in the same tissue of a human not afflicted with the bone-related disorder,
wherein a difference between
i) the level of expression of the mrr gene in the tissue of the patient and
ii) the level of expression of the mrr gene in the same tissue of the human not afflicted with the bone-related disorder
is an indication that the patient is afflicted with the bone-related disorder;
e) determining the MRR protein content of a tissue of the patient and
comparing
i) the MRR protein content of the tissue of the patient and
ii) the MRR protein content of the same tissue of a human not afflicted with the bone-related disorder,
wherein a difference between
i) the MRR protein content of the tissue of the patient and
ii) the MRR protein content of the same tissue of the human not afflicted with the bone-related disorder
is an indication that the patient is afflicted with the bone-related disorder;
f) administering a detectable MRR-binding agent to the patient,
detecting the amount of the MRR-binding agent in the patient, and
comparing
i) the amount of the MRR-binding agent in the patient and
ii) the amount of the MRR-binding agent in a human not afflicted with the bone-related disorder following administration of the MRR-binding agent thereto,
wherein a difference between
i) the amount of the MRR-binding agent in the patient and
ii) the amount of the MRR-binding agent in the human not afflicted with the bone-related disorder following administration of the MRR-binding agent thereto
is an indication that the patient is afflicted with the bone-related disorder; and
g) administering a detectable MRR-binding agent to the patient,
detecting the amount of the MRR-binding agent associated with a bone tissue in the patient, and
comparing
i) the amount of the MRR-binding agent associated with a bone tissue in the patient and
ii) the amount of the MRR-binding agent associated with a bone tissue in a human not afflicted with the bone-related disorder following administration of the MRR-binding agent thereto,
wherein a difference between
i) the amount of the MRR-binding agent associated with a bone tissue in the patient and
ii) the amount of the MRR-binding agent associated with a bone tissue in the human not afflicted with the bone-related disorder following administration of the MRR-binding agent thereto
is an indication that the patient is afflicted with the bone-related disorder.

2. The method of claim 1, wherein the bone-related disorder is selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue.

3. The method of claim 1, wherein the tissue of the patient is a bone tissue.

4. The method of claim 1, wherein the tissue of the patient is tissue sample obtained from the patient.

5. The method of claim 4, wherein the tissue sample is selected from the group consisting of a bone tissue biopsy, a blood fluid, and a cerebrospinal fluid.

6. The method of claim 1, wherein the human not afflicted with the bone-related disorder is a consensus profile of humans not afflicted with the bone-related disorder.

7. The method of claim 1, wherein the transcript polynucleotide is an mRNA.

8. The method of claim 1, wherein the transcript polynucleotide is a cDNA.

9. The method of claim 8, wherein the portion of the cDNA is the entire open reading frame corresponding to the cDNA

10. The method of claim 1, wherein the MRR-binding agent is an antibody substance.

11. The method of claim 10, wherein the antibody substance is attached to an imaging agent.

12. The method of claim 11, wherein the amount of the MRR-binding agent is detected using an imaging method in which the imaging agent can be detected.

13. The method of claim 1, wherein the mrr gene listed in GenBank Accession No. AF003625 includes nucleotide residues 27599 through 32411 of GenBank Accession No. AF003625.

14. The method of claim 1, wherein the portion of the MRR protein is the entire amino acid sequence of the MRR protein.

15. A method of determining whether an individual is predisposed to become afflicted with a bone-related disorder, the method selected from the group consisting of
a) determining the nucleotide sequence of a portion of an mrr gene of the patient and
comparing
i) the nucleotide sequence of the portion and
ii) the corresponding portion of the mrr gene listed in GenBank Accession No. AF003625,
wherein a difference between
i) the nucleotide sequence of the portion and
ii) the corresponding portion of GenBank Accession No. AF003625 is an indication that the patient is predisposed to become afflicted with the bone-related disorder;
b) determining the nucleotide sequence of a portion of a transcript polynucleotide of a tissue of the patient, wherein the transcript polynucleotide corresponds to an mrr gene of the patient and
comparing
i) the nucleotide sequence of the portion and
ii) the corresponding portion of SEQ ID NO: 2,
wherein a difference between
i) the nucleotide sequence of the portion and
ii) the corresponding portion of SEQ ID NO: 2
is an indication that the patient is predisposed to become afflicted with the bone-related disorder; and
c) determining the amino acid sequence of a portion of an MRR protein of a tissue of the patient and
comparing
i) the amino acid sequence of the portion of the MRR protein of the tissue of the patient and
ii) the corresponding portion of SEQ ID NO: 1,
wherein a difference between
i) the amino acid sequence of the portion of the MRR protein of the tissue of the patient and
ii) the corresponding portion of SEQ ID NO: 1
is an indication that the patient is predisposed to become afflicted with the bone-related disorder.

16. The method of claim 15, wherein the bone-related disorder is selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue.

17. A method of alleviating a bone-related disorder in a human patient, the method selected from the group consisting of:
a) providing a biologically active portion of an MRR protein to a bone tissue of the patient in an amount sufficient to alleviate the bone-related disorder;
b) providing a non-functional MRR protein to a bone tissue of the patient in an amount sufficient to alleviate the bone-related disorder;
c) providing an expression vector comprising a polynucleotide encoding a biologically active portion of an MRR protein to a bone tissue of the patient in an amount sufficient to alleviate the bone-related disorder;
d) providing an expression vector comprising a polynucleotide encoding a non-functional MRR protein to a bone tissue of the patient in an amount sufficient to alleviate the bone-related disorder;
e) providing an agonist of MRR protein activity to a bone tissue of the patient in an amount sufficient to alleviate the bone-related disorder;
f) providing an antagonist of MRR protein activity to a bone tissue of the patient in an amount sufficient to alleviate the bone-related disorder;
g) providing an enhancer of mrr expression to a bone tissue of the patient in an amount sufficient to alleviate the bone-related disorder; and
h) providing an inhibitor of mrr expression to a bone tissue of the patient in an amount sufficient to alleviate the bone-related disorder.

18. The method of claim 17, wherein the bone-related disorder is selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue.

19. The method of claim 17, wherein the biologically active portion of the MRR protein is a protein having the sequence SEQ ID NO: 1.

20. The method of claim 17, wherein the polynucleotide encoding a biologically active portion of an MRR protein is a polynucleotide having the sequence SEQ ID NO: 2.

21. The method of claim 17, wherein the antagonist of MRR protein activity is an antibody substance which binds specifically with MRR protein.

22. The method of claim 17, wherein the inhibitor of mrr expression is selected from the group consisting of an anti-sense oligonucleotide and a ribozyme.

23. A method of inhibiting a bone-related disorder in a human patient at risk for developing the bone-related disorder, the method selected from the group consisting of:
a) providing a biologically active portion of an MRR protein to a bone tissue of the patient in an amount sufficient to inhibit the bone-related disorder;
b) providing a non-functional MRR protein to a bone tissue of the patient in an amount sufficient to inhibit the bone-related disorder;
c) providing an expression vector comprising a polynucleotide encoding a biologically active portion of an MRR protein to a bone tissue of the patient in an amount sufficient to inhibit the bone-related disorder;
d) providing an expression vector comprising a polynucleotide encoding a non-functional MRR protein to a bone tissue of the patient in an amount sufficient to inhibit the bone-related disorder;
e) providing an agonist of MRR protein activity to a bone tissue of the patient in an amount sufficient to inhibit the bone-related disorder;
f) providing an antagonist of MRR protein activity to a bone tissue of the patient in an amount sufficient to inhibit the bone-related disorder;
g) providing an enhancer of mrr expression to a bone tissue of the patient in an amount sufficient to inhibit the bone-related disorder; and
h) providing an inhibitor of mrr expression to a bone tissue of the patient in an amount sufficient to inhibit the bone-related disorder.

24. The method of claim 23, wherein the bone-related disorder is selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue.

25. A method of determining whether a test composition is useful for alleviating a bone-related disorder, the method selected from the group consisting of:
a) maintaining a cell which comprises a biologically active MRR protein in the presence of the test composition and
comparing
i) an activity of the MRR protein of the cell maintained in the presence of the test composition and
ii) the same activity of the MRR protein of a cell of the same type maintained in the absence of the test composition,
wherein a difference between
i) an activity of the MRR protein of the cell maintained in the presence of the test composition and
ii) the same activity of the MRR protein of the cell of the same type maintained in the absence of the test composition
is an indication that the test composition is useful for alleviating a bone-related disorder;
b) maintaining a cell which comprises a biologically active MRR protein in the presence of the test composition and
comparing
i) the level of expression of the mrr gene in the cell maintained in the presence of the test composition and
ii) the level of expression of the mrr gene in the MRR protein of a cell of the same type maintained in the absence of the test composition,
wherein a difference between
i) the level of expression of the mrr gene in the cell maintained in the presence of the test composition and
ii) the level of expression of the mrr gene in the MRR protein of a cell of the same type maintained in the absence of the test composition
is an indication that the test composition is useful for alleviating a bone-related disorder;
c) maintaining a cell which comprises a biologically active MRR protein in the presence of the test composition and
comparing
i) a bone phenotype of the cell maintained in the presence of the test composition and
ii) the same bone phenotype of a cell of the same type maintained in the absence of the test composition,
wherein a difference between
i) the bone phenotype of the cell maintained in the presence of the test composition and
ii) the same bone phenotype of the cell maintained in the absence of the test composition
is an indication that the test composition is useful for alleviating a bone-related disorder;
d) administering the test composition to a first animal which naturally harbors an mrr gene and
comparing
i) a bone phenotype of the first transgenic animal and
ii) the same bone phenotype of a second animal which naturally harbors an mrr gene and to which the test composition is not administered,
wherein a difference between
i) the bone phenotype of the first animal and
ii) the same bone phenotype of the second animal
is an indication that the test composition is useful for alleviating a bone-related disorder;
e) administering the test composition to a first non-human transgenic animal which harbors an exogenous mrr gene and
comparing
i) a bone phenotype of the first transgenic animal and
ii) the same bone phenotype of a second non-human transgenic animal which harbors an exogenous mrr gene and to which the test composition is not administered,
wherein a difference between
i) the bone phenotype of the first transgenic animal and
ii) the same bone phenotype of the second transgenic animal
is an indication that the test composition is useful for alleviating a bone-related disorder; and
f) maintaining an artificial membrane which comprises a biologically active MRR protein in the presence of the test composition and
comparing
i) an activity of the MRR protein of the artificial membrane maintained in the presence of the test composition and
ii) the same activity of the MRR protein of an artificial membrane of the same type maintained in the absence of the test composition,
wherein a difference between
i) the activity of the MRR protein of the artificial membrane maintained in the presence of the test composition and
ii) the same activity of the MRR protein of artificial membrane of the same type maintained in the absence of the test composition
is an indication that the test composition is useful for alleviating a bone-related disorder.

26. The method of claim 25, wherein the bone-related disorder is selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue.

27. The method of claim 25, wherein the biologically active MRR protein is a protein having the amino acid sequence SEQ ID NO: 1.

28. The method of claim 25, wherein the activity of the MRR protein is selected from the group consisting of a proteolytic activity, a pore-modulating activity, an enzyme-modulating activity, and a gene transcription-modulating activity.

29. The method of claim 25, wherein the cell is an animal cell.

30. The method of claim 29, wherein the cell is a bone cell.

31. The method of claim 30, wherein the animal cell is selected from the group consisting of a human cell, a mouse cell, and a rat cell.

32. The method of claim 25, wherein the mrr gene is selected from the group consisting of a human mrr gene, a mouse mrr gene, and a rat mrr gene.

33. The method of claim 32, wherein the mrr gene encodes a protein having an amino acid sequence selected from the group consisting of SEQ ID NOs: 1 and 3.

34. The method of claim 25, wherein the bone phenotype is selected from the group consisting of a bone deposition phenotype, a bone resorption phenotype, and a bone morphology phenotype.

35. The method of claim 25, wherein the exogenous mrr gene encodes a protein having the amino acid sequence SEQ ID NO: 1.

36. The method of claim 25, wherein the artificial membrane is selected from the group consisting of a liposome and a re-sealed erythrocyte.

37. A method of determining the propensity of a test compound to induce a bone-related disorder in a human patient, the method selected from the group consisting of:
a) maintaining a cell which comprises a biologically active MRR protein in the presence of the test composition and
comparing
i) an activity of the MRR protein of the cell maintained in the presence of the test composition and
ii) the same activity of the MRR protein of a cell of the same type maintained in the absence of the test composition,
wherein a difference between
i) an activity of the MRR protein of the cell maintained in the presence of the test composition and
ii) the same activity of the MRR protein of the cell of the same type maintained in the absence of the test composition
is an indication that the test composition is likely to induce the bone-related disorder in a human patient;
b) maintaining a cell which comprises a biologically active MRR protein in the presence of the test composition and
comparing
i) the level of expression of the mrr gene in the cell maintained in the presence of the test composition and
ii) the level of expression of the mrr gene in the MRR protein of a cell of the same type maintained in the absence of the test composition,
wherein a difference between
i) the level of expression of the mrr gene in the cell maintained in the presence of the test composition and
ii) the level of expression of the mrr gene in the MRR protein of a cell of the same type maintained in the absence of the test composition
is an indication that the test composition is likely to induce the bone-related disorder in a human patient;
c) maintaining a cell which comprises a biologically active MRR protein in the presence of the test composition and
comparing
i) a bone phenotype of the cell maintained in the presence of the test composition and
ii) the same bone phenotype of a cell of the same type maintained in the absence of the test composition,
wherein a difference between
i) the bone phenotype of the cell maintained in the presence of the test composition and
ii) the same bone phenotype of the cell maintained in the absence of the test composition
is an indication that the test composition is likely to induce the bone-related disorder in a human patient;
d) administering the test composition to a first animal which naturally harbors an mrr gene and
comparing
i) a bone phenotype of the first transgenic animal and
ii) the same bone phenotype of a second animal which naturally harbors an mrr gene and to which the test composition is not administered,
wherein a difference between
i) the bone phenotype of the first animal and
ii) the same bone phenotype of the second animal
is an indication that the test composition is likely to induce the bone-related disorder in a human patient;
e) administering the test composition to a first non-human transgenic animal which harbors an exogenous mrr gene and
comparing
i) a bone phenotype of the first transgenic animal and
ii) the same bone phenotype of a second non-human transgenic animal which harbors an exogenous mrr gene and to which the test composition is not administered,
wherein a difference between
i) the bone phenotype of the first transgenic animal and
ii) the same bone phenotype of the second transgenic animal is an indication that the test composition is likely to induce the bone-related disorder in a human patient; and
f) maintaining an artificial membrane which comprises a biologically active MRR protein in the presence of the test composition and
comparing
i) an activity of the MRR protein of the artificial membrane maintained in the presence of the test composition and
ii) the same activity of the MRR protein of an artificial membrane of the same type maintained in the absence of the test composition,
wherein a difference between
i) an activity of the MRR protein of the artificial membrane maintained in the presence of the test composition and
ii) the same activity of the MRR protein of artificial membrane of the same type maintained in the absence of the test composition
is an indication that the test composition is likely to induce the bone-related disorder in a human patient.

38. The method of claim 37, wherein the bone-related disorder is selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue.

39. A method of identifying a polymorphism associated with an mrr gene of a human patient afflicted with a bone-related disorder, the method comprising
determining the nucleotide sequence of a polynucleotide associated with the mrr gene in the patient and
comparing
i) the nucleotide sequence of a polynucleotide associated with the mrr gene in the patient and
ii) the nucleotide sequence of the corresponding portion of an mrr gene of a human not afflicted with the bone-related disorder,
wherein a difference between
i) the nucleotide sequence of the polynucleotide and
ii) the nucleotide sequence of the corresponding portion of an mrr gene of the human not afflicted with the bone-related disorder
indicates a polymorphism associated with the mrr gene of the human patient afflicted with the bone-related disorder.

40. The method of claim 39, wherein the bone-related disorder is selected from the group consisting of osteoporosis, Paget's disease, hyperthyroidism, hyperparathyroidism, osteomalacia, chronic renal failure, Cushing's syndrome, an osteogenic cancer, and a non-osteogenic cancer that has metastasized to bone tissue.

41. A method of determining whether a human patient is predisposed to become afflicted with a bone-related disorder, the method comprising determining whether a polymorphism identified by the method of claim 39 occurs in an mrr gene of the human patient, wherein occurrence of the polymorphism in the mrr gene of the human patient is an indication that the human patient is predisposed to become afflicted with the bone-related disorder.


SEQUENCE LISTING (9 sequences)

SEQ ID NO: 1 - protein, 613 aa, Homo sapiens
MGPTLAVPTPYGCIGCKLPQPEYPPALIIFMFCAMVITIVVDLIGNSMVILAVTKNKKLRNSGNIFVVSLSVADMLVAIYPYPLMLHAMSIGGWDLSQLQCQMVGFITGLSVVGSIFNIVAIAINRYCYICHSLQYERIFSVRNTCIYLVITWIMTVLAVLPNMYIGTIEYDPRTYTCIFNYLNNPVFTVTIVCIHFVLPLLIVGFCYVRIWTKVLAARDPAGQNPDNQLAEVRNFLTMFVIFLLFAVCWCPINVLTVLVAVSPKEMAGKIPNWLYLAAYFIAYFNSCLNAVIYGLLNENFRREYWTIFHAMRHPIIFFPGLISDIREMQEARTLARARAHARDQAREQDRAHACPAVEETPMNVRNVPLPGDAAAGHPDRASGHPKPHSRSSSAYRKSASTHHKSVFSHSKAASGHLKPVSGHSKPASGHPKSATVYPKPASVHFKGDSVHFKGDSVHFKPDSVHFKPASSNPKPITGHHVSAGSHSKSAFSAATSHPKPIKPATSHAEPTTADYPKPATTSHPKPAAADNPELSASHCPEIPAIAHPVSDDSDLPESASSPAAGPTKPAASQLESDTIADLPDPTVVTTSTNDYHDVVVVDVEDDPDEMAV

SEQ ID NO: 2 - DNA, 1939 bp, Homo sapiens
tgtttgctgtctggacctggctgctgatcctgagcctgctgggagatcttaacgatccccaggagcaacatggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttccctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaagggtgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccatcaagccagctaccagccatgctgagcccaccactgctgactatcccaagcctgccactaccagccaccctaagcccgctgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttgttgatgttgaagatgatcctgatgaaatggctgtgtgaaaaatgctctcgtaggtggccaggcagt

SEQ ID NO: 3 - protein, 591 aa, Mus sp.
MATVPKSNMGPTKAVPTPFGCIGCKLPKPDYPPALIIFMFCAMVITVVVDLIGNSMVILAVTKNKKLRNSGNIFVASLSVADMLVAIYPYPLMLYAMSVGGWDLSQLQCQMVGLVTGLSVVGSIFNITAIAINRYCYICHSLQYKRIFSLRNTCIYLVVTWVMTVLAVLPNMYIGTIEYDPRTYTCIFNYVNNPAFTVTIVCIHFVLPLIIVGYCYTKIWIKVLAARDPAGQNPDNQFAEVRNFLTMFVIFLLFAVCWCPVNVLTVLVAVIPKEMAGKIPNWLYLAAYCIAYFNSCLNAIIYGILNESFRREYWTIFHAMRHPILFISHLISDIRETWETRALTRARVRARDQVREQERARACVAVEGTPRNVRNVLLPGDASAPHSDRASVRPKPQTRSTSVYRKPASIHHKSISGHPKSASVYPKPASSVHCKPASVHFKPASVHFKGDSVYFKGDTVHYRAASKLVTSHRISAGPSTSHPTSMAGYIKSGTSHPATTTVDYLEPATTSHSVLTAVDLPEVSASHCLEMTSTGHLRADISASVLPSVPFELAATPPDTTAIPIASGDYRKVVLIDDDSDDSDCSDEMAV

SEQ ID NO: 4 - DNA, 1800 bp, Mus sp.
aagatcctgagcttgcctgggaggaatggccacggtccccaagagcaacatgggacctacaaaggcggttcccaccccattcggctgcattggctgtaagctgccaaagcccgactacccgccagctctaatcatcttcatgttctgcgcaatggtcatcacagtcgtcgtagacctgatcgggaactccatggtcattttggctgtgaccaagaacaagaagctccgaaattctggcaacatctttgtggccagcctctctgtggcagacatgctggtggccatctacccctaccctttgatgctgtatgccatgtcagttgggggctgggatctgagtcagctccagtgccagatggtcgggttggtcacaggactgagcgtagtcggttccatcttcaacattactgccattgccatcaaccgttactgctacatctgccacagcctccaatacaagcggatcttcagcctgcgcaacacttgcatctatctggtcgttacctgggtcatgactgtcctggctgtcctgcctaacatgtacattggcaccattgagtatgaccctcgcacctacacctgcatcttcaactatgtgaacaatcctgcctttaccgtgaccattgtctgcatccacttcgtcctccctctcatcatagttggttattgctacacgaaaatctggatcaaagtgctggcagcccgtgacccagctggacagaatcctgacaaccagtttgctgaggttcgaaattttctaaccatgtttgtgatcttcctcctttttgcagttgctggtgccctgtcaatgtgctcactgtgttggtggctgtcattccaaaggaaatggcaggcaagatccccaactggctttatcttgcagcctactgcatagcctacttcaacagctgcctcaacgccatcatctacggtatcctcaatgagagtttccgaagagaatactggaccatcttccatgctatgcggcaccctatcctgttcatctctcacctcatcagtgatattcgggagacttgggagacccgagctctcactcgtgcccgtgtccgtgcccgtgatcaagtccgagagcaagagcgtgctcgtgcctgtgtcgctgtggaggggaccccaaggaacgtccggaatgttctactgcctggtgatgcatcagcaccccactctgatcgtgcctctgtccgtcccaagccccaaaccaggtctacttctgtctaccgcaaacctgcctctatccaccacaagtctatttctggccaccccaagtctgcctctgtttaccctaagccagcctcctctgtccattgcaagcctgcctctgtccatttcaaacccgcctctgttcatttcaagggtgactctgtctatttcaagggagacactgtccattacagggctgcttccaaacttgtcaccagtcaccgtatctctgctggcccttccaccagtcaccctacatccatggctggctacattaaatctggtaccagccaccctgccaccaccactgttgactatctcgaacctgccaccaccagccactctgtgctcactgctgtcgacctccctgaggtctcagcctcccattgccttgagatgaccagcactggccacctcagagctgacatttctgcctctgtccttccttctgtacccttcgagcttgctgccacccctcctgataccactgcaatccccattgcctctggtgattaccgcaaggtcgtgcttattgatgatgattctgatgattctgattgctctgatgagatggctgtgtga

SEQ ID NO: 5 - protein, 119 aa, Rattus sp.
YCYICHSLQYNADLQPANTCIYLVVTWVMTVLDVLPNVYIGTIEYDPRTYTCYFNYVNNPAFTVTIVCIHFVLPLIIVGYCYTKIWIKVLADRDPAGQNPDNQFAEVRNFLTMFVIFLL

SEQ ID NO: 6 - DNA, 357 bp, Rattus sp.
tactgctacatctgccacagcctccagtacaatgcggatcttcagcctgctaacacttgcatctatctggttgttacctgggtcatgactgttctggatgtcctgcctaatgtgtacattggcaccattgagtatgaccctcgcacctacacctgctacttcaactatgtgaacaaccctgcctttactgtgaccattgtctgcatccacttcgtcctccctctcatcatagtcggttattgctacacaaaaatctggatcaaagtgctggcagaccgggacccagctggacagaatcctgacaaccagtttgctgaggttcgaaattttctaaccatgtttgtgatcttcctcctt

SEQ ID NO: 7 - DNA, 257 bp, Homo sapiens
tgtttgctgtctggacctggctgctgatcctgagcctgctgggagatcttaacgatccccaggagcaacatggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctgg

SEQ ID NO: 8 - DNA, 1320 bp, Homo sapiens
ttcaggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacc

SEQ ID NO: 9 - DNA, 368 bp, Homo sapiens
catcaagccagctaccagccatgctgagcccaccactgctgactatcccaagcctgccactaccagccaccctaagcccactgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttattgatgttgaagatgatcctgatgaaatggctgtgtgaaaaatgctctcgtaggtggccaggcagt